SEQUENCE LISTING (52 sequences)

SEQ ID NO: 1 - RNA, 22 nt, Caenorhabditis elegans
ucaccggguguaaaucagcuug

SEQ ID NO: 2 - RNA, 21 nt, Arabidopsis thaliana
uuuggauugaagggagcucua

SEQ ID NO: 3 - RNA, 22 nt, Homo sapiens
uagcagcacguaaauauuggcg

SEQ ID NO: 4 - RNA, 23 nt, Homo sapiens
caaagugcuuacagugcagguag

SEQ ID NO: 5 - RNA, 21 nt, Homo sapiens
uucaaguaauucaggauaggu

SEQ ID NO: 6 - RNA, 23 nt, Homo sapiens
agcagcauuguacagggcuauca

SEQ ID NO: 7 - RNA, 22 nt, Homo sapiens
ucccugagacccuaacuuguga

SEQ ID NO: 8 - RNA, 22 nt, Homo sapiens
uguaacagcaacuccaugugga

SEQ ID NO: 9 - RNA, 21 nt, Homo sapiens
agcuacaucuggcuacugggu

SEQ ID NO: 10 - RNA, 23 nt, Homo sapiens
ugaggggcagagagcgagacuuu

SEQ ID NO: 11 - RNA, 22 nt, Homo sapiens
cagcagcaauucauguuuugaa

SEQ ID NO: 12 - RNA, 22 nt, Homo sapiens
uacccauugcauaucggaguug

SEQ ID NO: 13 - RNA, 22 nt, Homo sapiens
ugagguaguagguuguauaguu

SEQ ID NO: 14 - RNA, 22 nt, Homo sapiens
agagguaguagguugcauaguu

SEQ ID NO: 15 - RNA, 22 nt, Homo sapiens
ugagguaggagguuguauaguu

SEQ ID NO: 16 - RNA, 22 nt, Homo sapiens
ugagguaguagauuguauaguu

SEQ ID NO: 17 - RNA, 22 nt, Homo sapiens
ugagguaguaguuugugcuguu

SEQ ID NO: 18 - DNA, 19 bp, Artificial Sequence
  sense primer for cel-miR39-3p
gtcaccgggtgtaaatcag

SEQ ID NO: 19 - DNA, 26 bp, Artificial Sequence
  antisense primer for cel-miR39-3p
ggtccagtttttttttttttttcaag

SEQ ID NO: 20 - DNA, 18 bp, Artificial Sequence
  sense primer for ath-miR159a
gcgcagtttggattgaag

SEQ ID NO: 21 - DNA, 27 bp, Artificial Sequence
  antisense primer for ath-miR159a
aggtccagtttttttttttttttagag

SEQ ID NO: 22 - DNA, 17 bp, Artificial Sequence
  sense primer for hsa-miR16-5p
cgcagtagcagcacgta

SEQ ID NO: 23 - DNA, 24 bp, Artificial Sequence
  antisense primer for hsa-miR16-5p
cagtttttttttttttttcgccaa

SEQ ID NO: 24 - DNA, 20 bp, Artificial Sequence
  sense primer for hsa-miR17-5p
gcaaagtgcttacagtgcag

SEQ ID NO: 25 - DNA, 26 bp, Artificial Sequence
  antisense primer for hsa-miR17-5p
ggtccagtttttttttttttttctac

SEQ ID NO: 26 - DNA, 22 bp, Artificial Sequence
  sense primer for hsa-miR26b-5p
cgcagttcaagtaattcaggat

SEQ ID NO: 27 - DNA, 26 bp, Artificial Sequence
  antisense primer for hsa-miR26b-5p
ggtccagtttttttttttttttacct

SEQ ID NO: 28 - DNA, 19 bp, Artificial Sequence
  sense primer for hsa-miR107-5p
gcagagcagcattgtacag

SEQ ID NO: 29 - DNA, 27 bp, Artificial Sequence
  antisense primer for hsa-miR107-5p
ggtccagtttttttttttttttgatag

SEQ ID NO: 30 - DNA, 17 bp, Artificial Sequence
  sense primer for hsa-miR125b-5p
gcagtccctgagaccct

SEQ ID NO: 31 - DNA, 26 bp, Artificial Sequence
  antisense primer for hsa-miR125b-5p
ccagtttttttttttttttcacaagt

SEQ ID NO: 32 - DNA, 19 bp, Artificial Sequence
  sense primer for hsa-miR194-5p
cagtgtaacagcaactcca

SEQ ID NO: 33 - DNA, 26 bp, Artificial Sequence
  antisense primer for hsa-miR194-5p
tccagtttttttttttttttccacat

SEQ ID NO: 34 - DNA, 18 bp, Artificial Sequence
  sense primer for hsa-miR222-3p
gcagagctacatctggct

SEQ ID NO: 35 - DNA, 26 bp, Artificial Sequence
  antisense primer for hsa-miR222-3p
ccagtttttttttttttttacccagt

SEQ ID NO: 36 - DNA, 17 bp, Artificial Sequence
  sense primer for hsa-miR423-5p
cagtgaggggcagagag

SEQ ID NO: 37 - DNA, 28 bp, Artificial Sequence
  antisense primer for hsa-miR423-5p
ggtccagtttttttttttttttaaagtc

SEQ ID NO: 38 - DNA, 18 bp, Artificial Sequence
  sense primer for hsa-miR424-5p
agcagcagcaattcatgt

SEQ ID NO: 39 - DNA, 26 bp, Artificial Sequence
  antisense primer for hsa-miR424-5p
aggtccagtttttttttttttttcaa

SEQ ID NO: 40 - DNA, 20 bp, Artificial Sequence
  sense primer for hsa-miR660-5p
agtacccattgcatatcgga

SEQ ID NO: 41 - DNA, 26 bp, Artificial Sequence
  antisense primer for hsa-miR660-5p
ggtccagtttttttttttttttcaac

SEQ ID NO: 42 - DNA, 19 bp, Artificial Sequence
  sense primer for hsa-let7a-5p
gcagtgaggtagtaggttg

SEQ ID NO: 43 - DNA, 30 bp, Artificial Sequence
  antisense primer for hsa-let7a-5p
ggtccagtttttttttttttttaactatac

SEQ ID NO: 44 - DNA, 20 bp, Artificial Sequence
  sense primer for hsa-let7d-5p
cgcagagaggtagtaggttg

SEQ ID NO: 45 - DNA, 29 bp, Artificial Sequence
  antisense primer for hsa-let7d-5p
ggtccagtttttttttttttttaactatg

SEQ ID NO: 46 - DNA, 19 bp, Artificial Sequence
  sense primer for hsa-let7e-5p
gcagtgaggtaggaggttg

SEQ ID NO: 47 - DNA, 30 bp, Artificial Sequence; identical to SEQ ID NO: 43
  antisense primer for hsa-let7e-5p

SEQ ID NO: 48 - DNA, 20 bp, Artificial Sequence
  sense primer for hsa-let7f-5p
cgcagtgaggtagtagattg

SEQ ID NO: 49 - DNA, 27 bp, Artificial Sequence
  antisense primer for hsa-let7f-5p
caggtccagtttttttttttttttaac

SEQ ID NO: 50 - DNA, 21 bp, Artificial Sequence
  sense primer for hsa-let7i-5p
gcagtgaggtagtagtttgtg

SEQ ID NO: 51 - DNA, 27 bp, Artificial Sequence
  antisense primer for hsa-let7i-5p
ggtccagtttttttttttttttaacag

SEQ ID NO: 52 - DNA, 26 bp, Artificial Sequence
  universal primer for reverse transcription (RT) reaction
  misc_feature (26)..(26): n is a, c, g, or t
caggtccagtttttttttttttttvn

(19) United States
(12) Patent Application Publication
(10) Pub. No.: US 2022/0325355 A1
(43) Pub. Date: Oct. 13, 2022

(54) METHOD FOR DIAGNOSING BREAST CANCER

(71) Applicant: Albert-Ludwigs-Universitaet Freiburg, Freiburg (DE)

(72) Inventors: Markus JAEGER, Ehrenkirchen (DE); Marc HIRSCHFELD, Walkenried (DE); Thalia ERBES, Gundelfingen (DE); Daniela WEISS, Emmendingen (DE)

(73) Assignee: Albert-Ludwigs-Universitaet Freiburg, Freiburg (DE)

(21) Appl. No.: 17/634,124

(22) PCT Filed: Aug. 7, 2020
(86) PCT No.: PCT/EP2020/072248

§ 371 Date: Feb. 9, 2022

(30) Foreign Application Priority Data
Aug. 9, 2019 (EP) 19190989.4

(51) Int. Cl. C12Q 1/6886; C12N 15/10; G01N 33/574; G01N 33/50; C12Q 1/6806

(52) U.S. Cl. CPC C12Q 1/6886; C12N 15/1017; G01N 33/57415; G01N 33/5005; C12Q 1/6806; C12Q 2600/178

(57) ABSTRACT

The invention relates to a method of diagnosing breast cancer and to the use of biomarkers for the detection and diagnosis of breast cancer.

### PRIORITY

This application corresponds to the U.S. National phase of International Application No. PCT/EP2020/072248, filed Aug. 7, 2020, which, in turn, claims priority to European Patent Application No. 19190989.4 filed Aug. 9, 2019, the contents of which are incorporated by reference herein in their entirety.

### SEQUENCE LISTING

The instant application contains a Sequence Listing that has been submitted in ASCII format via EFS-Web and is hereby incorporated by reference in its entirety. Said ASCII copy, created on Feb. 9, 2022, is named LNK 235US SeqList ST25.txt and is 10,026 bytes in size.

### BACKGROUND OF THE INVENTION

The distinct impact of breast cancer (BC) on female health and associated life expectancy is reflected by a continuously increasing number of global incidence rates during the course of the past decades. Ascertainment of cancer registry data of 102 countries in the period of 1990-2016 as part of the Global Burden of Disease (GBD) study [1] identified breast cancer (BC) as the most frequent malignant disease in women with 1.7 million worldwide cases in 2016 [2]. Malignancies originating from breast tissue account for 25% of all female cancers [3]. Since 1990 the BC incidence rates more than doubled in 60/102 countries, affecting both, developed and developing regions [2]. Early detection of BC is the decisive factor for the cure rate [4-6] although BC-associated mortality rates could be reduced in general due to improved screening and treatment options, heterogeneous quality pattern in different countries sustain a crucial weak point in BC management [2, 7]. Routine BC detection methods including palpation, mammography and ultrasound are characterized by various limitations, e.g. moderate sensitivity and specificity rates especially at denser mammary tissue, decreased patient compliance [8, 9]. Thus, there is a huge demand for methodical innovation for improved (early) diagnosis of breast cancer.

Liquid biopsy-based disease biomarkers offer a range of promising prospects as important prognostic, diagnostic and theranostic tools [10]. Various biomarker types are available in body liquids via minimal- or non-invasive sampling procedures [10]. Among others, circulating microRNA (miRNA/miR) molecules qualify as robust and reliable matrices in regard to detectability and specificity in disease diagnosis and monitoring [10, 11]. Recently, the feasibility of urinary miRs in breast cancer detection could be demonstrated [12]. Similarly, the applicability of urinary miRs with diagnostic features was proven also in other tumor types [10, 13-15]. Subsequent to the isolation of miRs from exosomes in urine specimen, expression analysis of these small nucleic acids provides distinct characteristic pattern to distinguish cancer patients from healthy controls [12, 14, 16].

Dysregulated expression of let-7-family members of miRNAs could be linked to breast carcinogenesis [17-22]. Distinct let-7 miRNAs bear the potential to be employed as prospective molecular markers in BC diagnostics and as therapeutic targets [22-25].

The functional involvement and putative diagnostic implications of miR-17 in BC could be demonstrated in several studies [26-29] with one pointing out the predictive potential in BC recurrence management [30]. Aberrant expression levels of miR-125 relate to BC tumorigenesis, within which it displays a functional correlation with the HER2-overexpressing BC subtype influencing the corresponding metastatic risk and prognosis [31-34]. Malignant breast tissue transformation was found accompanied by miR-222 activities regulating decisive signaling pathways in tumor progression [35-37]. Most interestingly, miR-222 potentially offers predictive power in hormone receptor-positive BC [38]. Exosome-mediated intercellular transfer of miR-222 was found associated with BC metastasis [39]. With focus on BC, the biomarker potential of miR-107 could be demonstrated in various functional studies, which also revealed its impact on BRCA1 activity and BC recurrence prediction in triple-negative BC (TNBC) [40-45]. Among others, the levels of circulating miR-107 were found correlated with lymph node metastasis and receptor status in BC patients [46]. Wnt/β-catenin signaling pathway in BC was found regulated by miR-194, thereby affecting cancer cell proliferation, migration and invasion [47]. Circulating miR-194 molecules could be linked to BC recurrence risk [48], but also exhibited a functional interrelation with the anti-HER2 agent trastuzumab [49].

The functional background of miR-423 and the crucial impact of altered SNPs (single nucleotide polymorphisms) in the miR-423 gene on BC biology became apparent in recent investigations [50, 51]. Another functional aspect is provided by the combined expression levels of miR-423 and miR-4417, which could differentiate 70.1% of hereditary and non-hereditary BCs [52].

Aside from cancer-relevant regulatory impact in basal-like BC [53], tumor suppressor activities influencing BC chemotherapy resistance could be described for miR-424 [54]. A serum-based BC biomarker study could identify a 3-miR signature comprising the expression levels of miR-424, miR-29c, and miR-199a with the highest diagnostic accuracy for distinguishing breast cancer patients from healthy controls [55]. Oncogenic effects could be linked to miR-660 expression, with triggering functions in BC proliferation, migration and anti-apoptotic activities [56]. A BC tissue-derived next generation sequencing analysis extracted miR-660 as one promising biomarker candidate with significant prognostic features in overall survival (OS) and recurrence-free survival (RFS) in BC patients [57]. In this study, expression levels of a set of thirteen distinct miRNA types was analyzed in the urine of untreated BC patients compared to healthy controls in order to extract a non-invasive biomarker tool applicable for BC diagnosis.

EP 3070178 A1 describes studies on urine samples of breast cancer patients. It was found that the urinary miRNAs miR-21, miR-125b, miR-375 and miR-451 displayed significantly decreased expression levels in breast cancer patients compared to healthy controls. In addition, the urinary miRNA miR-155 showed an increased expression level in breast cancer patients. EP 3070178 A1 suggests that in particular determination of the expression level of the four miRNAs miR-21, miR-125b, miR-155 and miR-451 in urine samples allows for a discrimination between healthy individuals and BC patients.

There is an ongoing need for improved methods of diagnosing breast cancer.

### SUMMARY OF THE INVENTION

The inventors investigated urine samples of breast cancer patients and surprisingly found that certain miRNA types and combinations thereof are useful in diagnosing breast cancer. In particular, a panel of miRNA types, comprising at least miR-424, miR-660, and Iet7-i, was found to be a highly specific combinatory biomarker tool in discrimination of breast cancer patients vs. healthy controls based on urine specimen. The inventors further developed a highly efficient method for isolating exosomes from urine and other body fluids, which is particularly useful in the method for diagnosing BC described herein.

The present invention therefore relates to the following embodiments.

[1] A method of diagnosing whether a subject has cancer (e.g. breast cancer), comprising determining the level of at least one miR gene product in a urine sample from the subject, wherein the at least one miR gene product is selected from the group consisting of miR-424, miR-423, miR-660, let7-i, miR-17, let7-f, miR-222, miR-194, miR-125b, let7-d and combinations thereof, optionally wherein an alteration of the level of the respective miR gene product in the urine sample, relative to the level of the respective miR gene product in a control sample, is indicative of the subject having cancer.

[2] A method of diagnosing whether a subject has cancer (e.g. breast cancer), comprising determining the level of at least one miR gene product in a urine sample from the subject, wherein the at least one miR gene product is selected from the group consisting of miR-424, miR-660, let7-i and combinations thereof, wherein an alteration in the level of miR-424, miR-660 and/or let7-i in the urine sample, relative to the level of the respective miR gene product in a control sample, is indicative of the subject having cancer; optionally wherein a decrease in the level of miR-660, and/or let7-i in the urine sample, and/or an increase in the level of miR-424 in the urine sample, relative to the level of the respective miR gene product in a control sample, is indicative of the subject having cancer.

[3] The method of item [1] or [2], comprising determining the level of miR-424, miR-660 and let7-i in the urine sample, wherein an alteration in the level of miR-424, miR-660 and let7-i in the urine sample, relative to the level of the respective miR gene products in the control sample, is indicative of the subject having cancer; optionally wherein a decrease in the level of miR-660 and let7-i in the urine sample, and an increase in the level of miR-424 in the urine sample, relative to the level of the respective miR gene products in the control sample, is indicative of the subject having cancer.

[4] The method of item [3], further comprising determining the level of miR-423 in the urine sample from the subject, wherein an alteration in the level of miR-423, miR-424, miR-660 and let7-i in the urine sample, relative to the level of the respective miR gene products in the control sample, is indicative of the subject having cancer; optionally wherein a decrease in the level of miR-423, miR-660, and let7-i in the urine sample, and an increase in the level of miR-424 in the urine sample, relative to the level of the respective miR gene products in the control sample, is indicative of the subject having cancer.

[5] The method of item [3], further comprising determining the level of miR-125b in the urine sample from the subject, wherein an alteration in the level of miR-125b, miR-424, miR-660 and let7-i in the urine sample, relative to the level of the respective miR gene products in the control sample, is indicative of the subject having cancer; optionally wherein a decrease in the level of miR-660 and let7-i in the urine sample, and an increase in the level of miR-424 and miR-125b in the urine sample, relative to the level of the respective miR gene products in the control sample, is indicative of the subject having cancer.

[6] The method of item [3], further comprising determining the level of let7-d in the urine sample from the subject, wherein an alteration in the level of let7-d, miR-424, miR-660 and let7-i in the urine sample, relative to the level of the respective miR gene products in the control sample, is indicative of the subject having cancer; optionally wherein a decrease in the level of miR-660 and let7-i in the urine sample, and an increase in the level of miR-424 and let7-d in the urine sample, relative to the level of the respective miR gene products in the control sample, is indicative of the subject having cancer.

[7] The method of any one of the preceding items, further comprising determining the level of miR-17 in the urine sample from the subject, wherein an alteration in the level of miR-17, relative to the level of the miR-17 in the control sample, is indicative of the subject having cancer; optionally wherein an increase in the level of miR-17 in the urine sample, relative to the level of miR-17 in the control sample, is indicative of the subject having cancer.

[8] The method of any one of the preceding items, further comprising determining the level of let7-f in the urine sample from the subject, wherein an alteration in the level of let7-f, relative to the level of the miR-let7-f in the control sample, is indicative of the subject having cancer; optionally wherein a decrease in the level of let7-f in the urine sample, relative to the level of let7-f in the control sample, is indicative of the subject having cancer.

[9] The method of any one of the preceding items, further comprising determining the level of miR-222 in the urine sample from the subject, wherein an alteration in the level of miR-222, relative to the level of the miR-222 in the control sample, is indicative of the subject having cancer; optionally wherein an increase in the level of miR-222 in the urine sample, relative to the level of miR-222 in the control sample, is indicative of the subject having cancer.

[10] The method of any one of the preceding items, further comprising determining the level of miR-194 in the urine sample from the subject, wherein an alteration in the level of miR-194, relative to the level of the miR-194 in the control sample, is indicative of the subject having cancer; optionally wherein an increase in the level of miR-194 in the urine sample, relative to the level of miR-194 in the control sample, is indicative of the subject having cancer.

[11] A method of diagnosing whether a subject has cancer (e.g. breast cancer), comprising
a) determining in a urine sample from the subject the level of miR-424, miR-660 and/or let7-i,
b) comparing the level of miR-424 in the urine sample to a control level of miR-424, comparing the level of miR-660 in the urine sample to a control level of miR-660, and/or comparing the level of let7-i in the urine sample to a control level of let7-i; and
c) diagnosing whether the subject has cancer (e.g. breast cancer), wherein an alteration in the level of miR-424, miR-660 and/or let7-i in the urine sample, relative to the control level of the respective gene product is indicative of the subject having cancer (e.g. breast cancer); optionally wherein (i) a decrease in the level of miR-660 in the urine sample, relative to the control level of miR-660, (ii) a decrease in the level of let7-i in the urine sample, relative to the control level of let7-i, and/or (iii) an increase in the level of miR-424 in the urine sample, relative to the control level of miR-424, is indicative of the subject having cancer (e.g. breast cancer).

[12] The method of item [11], comprising
a) determining in the urine sample from the subject a level of miR-424, miR-660 and let7-i,
b) comparing the level of miR-424 in the urine sample to a control level of miR-424, comparing the level of miR-660 in the urine sample to a control level of miR-660, and comparing the level of let7-i in the urine sample to a control level of let7-i; and
c) diagnosing whether the subject has cancer (e.g. breast cancer), wherein an alteration in the level of miR-424, miR-660 and let7-i in the urine sample, relative to the control level of the respective gene product is indicative of the subject having cancer (e.g. breast cancer); optionally wherein (i) a decrease in the level of miR-660 in the urine sample, relative to the control level of miR-660, (ii) a decrease in the level of let7-i in the urine sample, relative to the control level of let7-i, and (iii) an increase in the level of miR-424 in the urine sample, relative to the control level of miR-424, is indicative of the subject having cancer (e.g. breast cancer).

[13] The method of item [11] or [12], further comprising:
a) determining in the urine sample from the subject the level of miR-423, miR-125b or let7-d,
b) comparing the level of miR-423, miR-125b or let7-d in the urine sample to the control level of the respective miR gene product, and
c) diagnosing whether the subject has cancer (e.g. breast cancer), wherein an alteration in the level of miR-423, miR-125b or let7-d in the urine sample, relative to the control level of the respective gene product is indicative of the subject having cancer (e.g. breast cancer); optionally wherein an increase in the level of miR-125b or let7-d in the urine sample, relative to the respective control level of miR-125b or let7-d, or a decrease in the level of miR-423 in the urine sample, relative to the control level of miR-423, is indicative of the subject having cancer (e.g. breast cancer).

[14] The method of any one of items [11] to [13], further comprising:
a) determining in the urine sample from the subject the level of miR-17, let7-f, miR-222 or miR-194,
b) comparing the level of miR-17, let7-f, miR-222 or miR-194 in the urine sample to a control level of the respective miR gene product, and
c) diagnosing whether the subject has cancer (e.g. breast cancer), wherein an alteration in the level of miR-17, let7-f, miR-222 or miR-194 in the urine sample, relative to the control level of the respective gene product is indicative of the subject having cancer (e.g. breast cancer); optionally wherein an increase in the level of miR-17, miR-222 or miR-194 in the urine sample, relative to the respective control level of miR-17, miR-222 or miR-194, or a decrease in the level of let7-f, relative to the control level of let7-f, is indicative of the subject having cancer (e.g. breast cancer).

[15] The method of any one of the preceding items, wherein the control sample is a urine sample from one or more healthy individuals, preferably from one or more healthy women.

[16] The method of any one of the preceding items, wherein the subject is a female human.

[17] The method of any one of the preceding items, wherein the level of miR gene product(s) is measured by quantitative RT-PCR, digital PCR, digital droplet PCR (ddPCR), next generation sequencing (NGS), by hybridisation using an oligonucleotide microarray, and/or by any other suitable technique.

[18] The method of any one of the preceding items, wherein said alteration in the level of miR gene product relative to the control level is at least 25%, or wherein said increase in the level of miR gene product relative to the control level is at least 25%, or wherein said decrease in the level of miR gene product relative to the control level is at least 25%.

[19] A kit for the detection or diagnosis of cancer (e.g. breast cancer), comprising an oligonucleotide capable of hybridizing to a nucleic acid consisting of SEQ ID NO:11, an oligonucleotide capable of hybridizing to a nucleic acid consisting of SEQ ID NO:12, and an oligonucleotide capable of hybridizing to a nucleic acid consisting of SEQ ID NO:17.

[20] The kit of item [19], further comprising an oligonucleotide capable of hybridizing to a nucleic acid consisting of SEQ ID NO:7, an oligonucleotide capable of hybridizing to a nucleic acid consisting of SEQ ID NO:10 and/or an oligonucleotide capable of hybridizing to a nucleic acid consisting of SEQ ID NO:14.

[21] The kit of item [19] or [20], further comprising an oligonucleotide capable of hybridizing to a nucleic acid consisting of SEQ ID NO:4, an oligonucleotide capable of hybridizing to a nucleic acid consisting of SEQ ID NO:8, an oligonucleotide capable of hybridizing to a nucleic acid consisting of SEQ ID NO:9 and/or an oligonucleotide capable of hybridizing to a nucleic acid consisting of SEQ ID NO:16.

[22] The kit of any one of items [19] to [21], comprising a pair of primers suitable for amplifying miR-424, miR-660 and let7-i.

[23] The kit of item [22], further comprising at least one pair of primers suitable for amplifying a miR gene product selected from the group consisting of miR-423, miR-17, let7-f, miR-222, miR-194, miR-125b, let7-d and combinations thereof.

[24] The kit of any one of items [19] to [23], further comprising a primer for the reverse transcription reaction.

[25] The use of an oligonucleotide capable of hybridizing to a nucleic acid consisting of SEQ ID NO:11, an oligonucleotide capable of hybridizing to a nucleic acid consisting of SEQ ID NO:12, and/or an oligonucleotide capable of hybridizing to a nucleic acid consisting of SEQ ID NO:17 for the diagnosis of cancer (e.g. breast cancer), said use comprising contacting said oligonucleotide(s) with a urine sample.

[26] The use of item [25], further comprising contacting an oligonucleotide capable of hybridizing to a nucleic acid consisting of SEQ ID NO:7, an oligonucleotide capable of hybridizing to a nucleic acid consisting of SEQ ID NO:10, and/or an oligonucleotide capable of hybridizing to a nucleic acid consisting of SEQ ID NO:14 with said urine sample.

[27] The use of item [25] or [26], further comprising contacting an oligonucleotide capable of hybridizing to a nucleic acid consisting of SEQ ID NO:4, an oligonucleotide capable of hybridizing to a nucleic acid consisting of SEQ ID NO:8, an oligonucleotide capable of hybridizing to a nucleic acid consisting of SEQ ID NO:9, and/or an oligonucleotide capable of hybridizing to a nucleic acid consisting of SEQ ID NO:16 with said urine sample.

[28] The kit or use of any one of items [19] to [27], wherein the oligonucleotide capable of hybridizing to a nucleic acid consisting of SEQ ID NO:11 is perfectly complementary to SEQ ID NO:11, the oligonucleotide capable of hybridizing to a nucleic acid consisting of SEQ ID NO:12 is perfectly complementary to SEQ ID NO:12, and/or the oligonucleotide capable of hybridizing to a nucleic acid consisting of SEQ ID NO:17 is perfectly complementary to SEQ ID NO:17.

[29] The kit or use of item [20] or [26], wherein said oligonucleotide capable of hybridizing to a nucleic acid consisting of SEQ ID NO:7 is perfectly complementary to SEQ ID NO:7, said oligonucleotide capable of hybridizing to a nucleic acid consisting of SEQ ID NO:10 is perfectly complementary to SEQ ID NO:10, and/or said oligonucleotide capable of hybridizing to a nucleic acid consisting of SEQ ID NO:14 is perfectly complementary to SEQ ID NO:14.

[30] The kit or use of item [21] or [27], wherein said oligonucleotide capable of hybridizing to a nucleic acid consisting of SEQ ID NO:4 is perfectly complementary to SEQ ID NO:4, said oligonucleotide capable of hybridizing to a nucleic acid consisting of SEQ ID NO:8 is perfectly complementary to SEQ ID NO:8, said oligonucleotide capable of hybridizing to a nucleic acid consisting of SEQ ID NO:9 is perfectly complementary to SEQ ID NO:9, and/or said oligonucleotide capable of hybridizing to a nucleic acid consisting of SEQ ID NO:16 is perfectly complementary to SEQ ID NO:16.

[31] The method, use or kit of any one of the preceding items, wherein the cancer is selected from the group consisting of breast carcinomas, lung carcinomas, gastric carcinomas, esophageal carcinomas, colorectal carcinomas, liver carcinomas, ovarian carcinomas, thecomas, arrhenoblastomas, cervical carcinomas, endometrial carcinoma, endometrial hyperplasia, endometriosis, fibrosarcomas, choriocarcinoma, head and neck cancer, nasopharyngeal carcinoma, laryngeal carcinomas, hepatoblastoma, Kaposi's sarcoma, melanoma, skin carcinomas, hemangioma, cavernous hemangioma, hemangioblastoma, pancreas carcinomas, retinoblastoma, astrocytoma, glioblastoma, Schwannoma, oligodendroglioma, medulloblastoma, neuroblastomas, rhabdomyosarcoma, osteogenic sarcoma, leiomyosarcomas, urinary tract carcinomas, thyroid carcinomas, Wilm's tumor, renal cell carcinoma, prostate carcinoma, abnormal vascular proliferation associated with phakomatoses, edema (such as that associated with brain tumors), and Meigs' syndrome.

[32] A method for enriching or isolating extracellular vesicles (e.g. microvesicles and/or exosomes) from a body fluid sample, e.g. a urine sample, comprising (i) optionally filtrating the body fluid sample (e.g. a urine sample) through a first membrane, wherein said first membrane has a pore size from 180 nm to 250 nm and does not bind proteins; (ii) filtrating the body fluid or, if step (i) is carried out, the filtrate obtained from step (i) through a second membrane, wherein said second membrane has a pore size from 180 nm to 250 nm and is a protein-binding membrane; and (iii) recovering the extracellular vesicles (e.g. microvesicles and/or exosomes) from the second membrane.

[33] The method of item [32], wherein the first membrane comprises, or substantially consists of, cellulose acetate.

[34] The method of item [32] or [33], wherein the second membrane comprises, or substantially consists of a polyamide, e.g. Nylon.

[35] The method of any one of items [32] to [34], wherein the pore size of the first membrane is about 0.20 μm, and/or wherein the pore size of the second membrane is about 0.22 μm.

[36] The method of any one of items [32] to [35], wherein the body fluid, e.g. urine, or the body fluid sample, is subjected to a centrifugation step prior to step (ii), or, if step (i) is carried out, prior to step (i).

[37] The method of item [36], wherein said centrifugation step comprises centrifuging the body fluid or the body fluid sample at 3,000 g to 5,000 g for 5 to 30 minutes, or at 3,500g to 4,500 g for 10 to 20 minutes, or at about 4,000 g for about 15 minutes.

[38] The method of any one of items [32] to [37],wherein the body fluid is blood, plasma, urine, ascites, saliva, or amniotic liquor.

[39] The method of any one of items [32] to [38], wherein filtration through the first membrane and/or the filtration through the second membrane is/are carried out using a syringe with screwed-on filters.

[40] The method of any one of items [32] to [39], wherein said recovering is effected by contacting the second membrane with a lysis buffer.

[41] The method of item [40], wherein said lysis buffer comprises at least one surfactant.

[42] The method of item [41], wherein said at least one surfactant is sodium lauryl sulfate (SDS) and/or polyethylene glycol p-(1,1,3,3-tetramethylbutyl)-phenyl ether (Triton X100).

[43] The method of any one of items [32] to [42], comprising separating microvesicles from exosomes, preferable by carrying out step (i).

[44] The method of any one of items [1] to [31], further comprising a method as defined in any one of items [32] to [43].

[45] The method of any one of items [1] to [31], wherein the urine sample is obtained by a method for enriching or isolating exosomes as defined in any one of [32] to [43].

[46] The method of any one of items [1] to [31], wherein the urine sample is provided by enriching or isolating exosomes by a method as defined in any one of [32] to [43].

[47] The method of any one of items [1] to [31], wherein the urine sample comprises, or substantially consists of, enriched or isolated exosomes obtainable or obtained by a method as defined in any one of [32] to [43].

### BRIEF DESCRIPTION OF THE DRAWINGS

FIG. 1: NRQ values of miR expression in urine specimen of BC patients.

FIG. 2: NRQ values of miR expression in urine specimen of healthy controls.

FIG. 3: Coefficient paths for the stepwise boosting procedure. The x-axis shows the boosting steps (here 1 to 1500), the y-axis shows the coefficient estimates for each selected variable. After 1500 steps, seven variables had been selected.

FIG. 4: Workflow scheme on microvesicle isolation from body fluids.

FIG. 5: Equipment assembly of filtration method.

FIG. 6: Western Blot showing the result of Example 2.

### DETAILED DESCRIPTION OF THE PREFERRED EMBODIMENTS

The present invention encompasses methods of diagnosing whether a subject has, or is at risk for developing, breast cancer, comprising determining the level of at least one miR gene product in a urine sample from the subject and comparing the level of the miR gene product in the urine sample to the level of the respective miR gene product in a control sample.

In particular, the invention relates to a method of diagnosing whether a subject has cancer (e.g. breast cancer), comprising determining the level of at least one miR gene product in a urine sample from the subject, wherein an increase or a decrease in the level of the miR gene product in the urine sample, relative to the level of the respective miR gene product in a control sample, is indicative of the subject having cancer (e.g. breast cancer), wherein the at least one miR gene product is selected from the group consisting of miR-424, miR-660, let7-i and combinations thereof.

As used herein interchangeably, a “miR gene product,” “microRNA,” “miR,” or “miRNA” refers to the unprocessed (e.g., precursor) or processed (e.g., mature) RNA transcript from a miR gene. As the miR gene products are not translated into protein, the term “miR gene products” does not include proteins. The unprocessed miR gene transcript is also called a “miR precursor” or “miR prec” and typically comprises an RNA transcript of about 70-100 nucleotides in length. The miR precursor can be processed by digestion with an RNAse (for example, Dicer, Argonaut, or RNAse III (e.g., E. coli RNAse III)) into an active 19-25 nucleotide RNA molecule. This active 19-25 nucleotide RNA molecule is also called the “processed” miR gene transcript or “mature” miRNA. The active 19-25 nucleotide RNA molecule can be obtained from the miR precursor through natural processing routes (e.g., using intact cells or cell lysates) or by synthetic processing routes (e.g., using isolated processing enzymes, such as isolated Dicer, Argonaut, or RNAse III). It is understood that the active 19-25 nucleotide RNA molecule can also be produced directly by biological or chemical synthesis, without having been processed from the miR precursor. When a microRNA is referred to herein by name, the name corresponds to both the precursor and mature forms, unless otherwise indicated. Table 1 depicts the nucleotide sequences of preferred mature human microRNAs used in the present invention.

TABLE 1

| Name of | | SEQ ID NO: of |
|---|---|---|
| microRNA | Mature sequence | mature sequence |
| miR-423 | UGAGGGGCAGAGAGCGAGACUUU | 10 |
| miR-424 | CAGCAGCAAUUCAUGUUUUGAA | 11 |
| miR-660 | UACCCAUUGCAUAUCGGAGUUG | 12 |
| let7-i | UGAGGUAGUAGUUUGUGCUGUU | 17 |
| miR-125b | UCCCUGAGACCCUAACUUGUGA | 7 |
| let7-d | AGAGGUAGUAGGUUGCAUAGUU | 14 |
| miR-17 | CAAAGUGCUUACAGUGCAGGUAG | 4 |
| let7-f | UGAGGUAGUAGAUUGUAUAGUU | 16 |
| miR194 | UGUAACAGCAACUCCAUGUGGA | 8 |
| miR222 | AGCUACAUCUGGCUACUGGGU | 9 |

As used herein, a “subject” can be any mammal that has, or is suspected of having, breast cancer. In a preferred embodiment, the subject is a human who has, or is suspected of having, breast cancer. Most preferably, the subject is a female human, e.g. a woman at an age of 30 to 70 years, 35 to 69 years, 40 to 67 years, or 45 to 65 years.

The term “urine sample” refers to a sample which comprises or consists of urine, or which is derived from urine. For determining the level of miR gene products in the urine sample, native urine is typically processed prior to analysis. The processing may include (but is not limited to) RNA isolation and/or purification, centrifugation, nucleic acid precipitation, dissolution of precipitated nucleic acid, concentration, dilution, filtration and combinations thereof. Such processed samples are encompassed by the term “urine sample”. In one embodiment the urine sample to be analysed is obtainable or obtained by enriching or isolating extracellular vesicles from urine. In another embodiment the urine sample is obtainable or obtained by enriching or isolating exosomes from urine. In preferred embodiments the urine sample to be analysed is obtainable or obtained by a method for isolating exosomes described hereinbelow.

As used herein, the term “extracellular vesicles” (EVs) refers to membrane-contained vesicles released by cells. EVs can be broadly classified into 3 main classes: (a) Microvesicles (or microparticles or ectosomes) that are produced by outward budding and fission of the plasma membrane; (b) Exosomes that are formed within the endosomal network and released upon fusion of multi-vesicular bodies with the plasma membrane; and (c) Apoptotic bodies which are released as blebs of cells undergoing apoptosis.

The level of at least one miR gene product is determined in a urine sample obtained from the subject. A corresponding control sample, which is also a urine sample, can be obtained from a healthy human individual or population of healthy individuals. The healthy “control” individual preferably has the same gender as the subject, and optionally a similar age (i.e. +/−10 years the age of the subject). Most preferably, the “control” individual is a healthy woman at an age of 30 to 70 years, 35 to 69 years, 40 to 67 years, or 45 to 65 years. The control urine sample is then processed along with the urine sample from the subject, so that the levels of miR gene product in the subject's urine sample can be compared to the respective miR gene product levels in the control sample. A reference miR expression standard for the urine sample can also be used as a control.

An alteration (e.g., an increase or decrease) in the level of a miR gene product in the sample obtained from the subject, relative to the level of the respective miR gene product in a control sample, as described below, is indicative of the presence of breast cancer in the subject. In one embodiment, the level of the at least one miR gene product in the test sample is greater than the level of the respective miR gene product in the control sample (i.e., expression of the miR gene product is “up-regulated”). As used herein, expression of a miR gene product is “up-regulated” when the amount of miR gene product in a urine sample from a subject is greater than the amount of the same gene product in a control sample.

In another embodiment, the level of the at least one miR gene product in the urine sample is less than the level of the respective miR gene product in the control sample (i.e., expression of the miR gene product is “down-regulated”). As used herein, expression of a miR gene is “down-regulated” when the amount of miR gene product produced from that gene in a urine sample from a subject is less than the amount produced from the same gene in a control sample.

The relative miR gene expression in the control and normal samples can be determined with respect to one or more RNA expression standards. The standards can comprise, for example, the urinary miR gene expression level in healthy subject, or the average level of urinary miR gene expression previously obtained for a population of healthy human controls. “Healthy” means that the subject or human controls do not have breast cancer or another type of cancer.

In one embodiment, the method comprises determining the level of a miR-424 gene product, wherein an alteration (e.g. an increase) in the level of the miR-424 gene product in the urine sample, relative to the level of miR-424 gene product in a control sample, is indicative of the subject having breast cancer. The miR-424 gene product preferably is an RNA molecule comprising or substantially consisting of SEQ ID NO:11, e.g. (i) an RNA molecule substantially consisting of SEQ ID NO:11, (ii) an RNA molecule consisting of SEQ ID NO:11, or (iii) an RNA molecule comprising SEQ ID NO:11.

In one embodiment, the method comprises determining the level of a miR-660 gene product, wherein an alteration (e.g. a decrease) in the level of the miR-660 gene product in the urine sample, relative to the level of miR-660 gene product in a control sample, is indicative of the subject having breast cancer. The miR-660 gene product preferably is an RNA molecule comprising or substantially consisting of SEQ ID NO:12, e.g. (i) an RNA molecule substantially consisting of SEQ ID NO:12, (ii) an RNA molecule consisting of SEQ ID NO:12, or (iii) an RNA molecule comprising SEQ ID NO:12.

In another embodiment, the method comprises determining the level of a let7-i gene product, wherein an alteration (e.g. a decrease) in the level of the let7-i gene product in the urine sample, relative to the level of let7-i gene product in a control sample, is indicative of the subject having breast cancer. The let7-i gene product preferably is an RNA molecule comprising or substantially consisting of SEQ ID NO:17, e.g. (i) an RNA molecule substantially consisting of SEQ ID NO:17, (ii) an RNA molecule consisting of SEQ ID NO:17, or (iii) an RNA molecule comprising SEQ ID NO:17.

In another embodiment, the method comprises determining the level of a miR-423 gene product, wherein an alteration (e.g. a decrease) in the level of the miR-423 gene product in the urine sample, relative to the level of miR-423 gene product in a control sample, is indicative of the subject having breast cancer. The miR-423 gene product preferably is an RNA molecule comprising or substantially consisting of SEQ ID NO:10, e.g. (i) an RNA molecule substantially consisting of SEQ ID NO:10, (ii) an RNA molecule consisting of SEQ ID NO:10, or (iii) an RNA molecule comprising SEQ ID NO:10.

In another embodiment, the method comprises determining the level of a let7-d gene product, wherein an alteration (e.g. an increase) in the level of the let7-d gene product in the urine sample, relative to the level of let7-d gene product in a control sample, is indicative of the subject having breast cancer. The let7-d gene product preferably is an RNA molecule comprising or substantially consisting of SEQ ID NO:14, e.g. (i) an RNA molecule substantially consisting of SEQ ID NO:14, (ii) an RNA molecule consisting of SEQ ID NO:14, or (iii) an RNA molecule comprising SEQ ID NO:14.

In another embodiment, the method comprises determining the level of a miR-125b gene product, wherein an alteration (e.g. an increase) in the level of the miR-125b gene product in the urine sample, relative to the level of miR-125b gene product in a control sample, is indicative of the subject having breast cancer. The miR-125b gene product preferably is an RNA molecule comprising or substantially consisting of SEQ ID NO:7, e.g. (i) an RNA molecule substantially consisting of SEQ ID NO:7, (ii) an RNA molecule consisting of SEQ ID NO:7, or (iii) an RNA molecule comprising SEQ ID NO:7.

In another embodiment, the method comprises determining the level of a let7-f gene product, wherein an alteration (e.g. a decrease) in the level of the let7-f gene product in the urine sample, relative to the level of let7-f gene product in a control sample, is indicative of the subject having breast cancer. The let7-f gene product preferably is an RNA molecule comprising or substantially consisting of SEQ ID NO:16, e.g. (i) an RNA molecule substantially consisting of SEQ ID NO:16, (ii) an RNA molecule consisting of SEQ ID NO:16, or (iii) an RNA molecule comprising SEQ ID NO:16.

In another embodiment, the method comprises determining the level of a miR-17 gene product, wherein an alteration (e.g. an increase) in the level of the miR-17 gene product in the urine sample, relative to the level of miR-17 gene product in a control sample, is indicative of the subject having breast cancer. The miR-17 gene product preferably is an RNA molecule comprising or substantially consisting of SEQ ID NO:4, e.g. (i) an RNA molecule substantially consisting of SEQ ID NO:4, (ii) an RNA molecule consisting of SEQ ID NO:4, or (iii) an RNA molecule comprising SEQ ID NO:4.

In another embodiment, the method comprises determining the level of a miR-222 gene product, wherein an alteration (e.g. an increase) in the level of the miR-222 gene product in the urine sample, relative to the level of miR-222 gene product in a control sample, is indicative of the subject having breast cancer. The miR-222 gene product preferably is an RNA molecule comprising or substantially consisting of SEQ ID NO:9, e.g. (i) an RNA molecule substantially consisting of SEQ ID NO:9, (ii) an RNA molecule consisting of SEQ ID NO:9, or (iii) an RNA molecule comprising SEQ ID NO:9.

In another embodiment, the method comprises determining the level of a miR-194 gene product, wherein an alteration (e.g. an increase) in the level of the miR-194 gene product in the urine sample, relative to the level of miR-194 gene product in a control sample, is indicative of the subject having breast cancer. The miR-194 gene product preferably is an RNA molecule comprising or substantially consisting of SEQ ID NO:8, e.g. (i) an RNA molecule substantially consisting of SEQ ID NO:8, (ii) an RNA molecule consisting of SEQ ID NO:8, or (iii) an RNA molecule comprising SEQ ID NO:8.

In another embodiment, the method comprises determining the level of a miR-424 gene product, a miR-660 gene product and a let7-i gene product, wherein a decrease in the levels of the let7-i and miR-660 gene products in the urine sample, relative to the respective levels of the respective miR gene products in a control sample, and an increase in the level of the miR-424 gene product in the urine sample, relative to the level of the miR-424 gene product in the control sample, is indicative of the subject having breast cancer.

In another embodiment, the method comprises determining the level of a miR-423 gene product, a miR-424 gene product, a miR-660 gene product and a let7-i gene product, wherein a decrease in the levels of the miR-423, mir-660 and let7-i gene products in the urine sample, relative to the respective levels of the respective miR gene products in a control sample, and an increase in the level of the miR-424 gene product in the urine sample, relative to the level of the miR-424 gene product in the control sample, is indicative of the subject having breast cancer.

In another embodiment, the method comprises determining the level of a miR-125b gene product, a miR-424 gene product, a miR-660 gene product and a let7-i gene product, wherein a decrease in the levels of the mir-660 and let7-i gene products in the urine sample, relative to the respective levels of the respective miR gene products in a control sample, and an increase in the levels of the miR-424 and miR-125b gene products in the urine sample, relative to the respective levels of the respective gene products in the control sample, is indicative of the subject having breast cancer.

In another embodiment, the method comprises determining the level of a let7-d gene product, a miR-424 gene product, a miR-660 gene product and a let7-i gene product, wherein a decrease in the levels of the mir-660 and let7-i gene products in the urine sample, relative to the respective levels of the respective miR gene products in a control sample, and an increase in the levels of the miR-424 and let7-d gene products in the urine sample, relative to the respective levels of the respective gene products in the control sample, is indicative of the subject having breast cancer.

### Determining the Level of miR Gene Products:

The level of a miR gene product in a urine sample can be measured using any technique that is suitable for detecting RNA expression levels in a urine sample. Nucleic acids can used be as probes or primers for embodiments involving nucleic acid hybridization. As such, they may be used to assess miRNA expression. The nucleotide sequences of the invention may be used for their ability to selectively form duplex molecules with complementary stretches of DNAs and/or RNAs or to provide primers for amplification of DNA or RNA from samples.

Preferably the processing efficiency in regard to RNA extraction, complementary DNA synthesis (reverse transcription) and PCR amplification is monitored by addition of “spike-in” control RNA specimen of defined concentration to the RNA lysis buffer as recommended by Marabita [4]. The detailed procedure is described herein in section 1.6 of Example 1.

In another preferred embodiment the amount of each miR gene product is normalized by converting it into normalized relative quantities (NRQ) for the respective miR gene product. In that embodiment the NRQ is used as the level of miR gene product.

Reverse transcription (RT) of RNA to cDNA followed by quantitative PCR (RT-PCR) can be used to determine the relative concentrations of specific miRNA species. By determining that the concentration of a specific mRNA species varies, it is shown that the gene encoding the specific mRNA species is differentially expressed. Another method for amplification is ligase chain reaction (“LCR”). U.S. Pat. No. 4,883,750 describes a method similar to LCR for binding probe pairs to a target sequence. A method based on PCR™ and oligonucleotide ligase assay (OLA), disclosed in U.S. Pat. No. 5,912,148, may also be used. Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety. Alternatively, digital PCT or digital droplet PCR or next generation sequencing can be used to determine the level of miR gene product in the urine sample. Any other suitable method for determining the amount of micro RNA can be used.

Alternatively, an oligolibrary, in microchip format (i.e., a microarray), may be constructed containing a set of oligonucleotide probes that are specific for a set of miR genes. Using such a microarray, the expression level of multiple microRNAs in a urine sample can be determined by reverse transcribing the RNAs to generate a set of target oligodeoxynucleotides, and hybridizing them to probe the oligonucleotides on the microarray to generate a hybridization, or expression, profile. The hybridization profile of the test sample can then be compared to that of a control sample to determine which microRNAs have an altered expression level in breast cancer cells. As used herein, “probe oligonucleotide” or “probe oligodeoxynucleotide” refers to an oligonucleotide that is capable of hybridizing to a target oligonucleotide. “Target oligonucleotide” or “target oligodeoxynucleotide” refers to a molecule to be detected (e.g., via hybridization). By “miR-specific probe oligonucleotide” or “probe oligonucleotide specific for a miR” is meant a probe oligonucleotide that has a sequence selected to hybridize to a specific miR gene product, or to a reverse transcript of the specific miR gene product.

The method of the invention may further comprise the step of comparing the level of the miR gene product in the urine sample to a control level of the miR gene product (e.g. the level in a control sample), and diagnosing whether the subject has breast cancer, wherein a decrease or increase of miR gene product in the urine sample, relative to the control level of miR gene product is indicative of the subject having breast cancer.

Breast cancer is a cancer that starts in the breast, usually in the inner lining of the milk ducts or lobules. There are different types of breast cancer, with different stages (spread), aggressiveness, and genetic makeup. Breast cancer subtypes may be categorized on an immunohistochemical basis. The breast cancer to be diagnosed, detected, monitored or screened in accordance with this invention may be invasive ductal carcinoma, ductal carcinoma in situ, or invasive lobular carcinoma. Alternatively, BC may be classified on the basis of receptor status. In another embodiment, the breast cancer to be diagnosed, detected, monitored or screened in accordance with this invention may therefore be:
“normal” BC (ER+, PR+, HER2+, cytokeratin 5/6+, and HER1+),
“luminal A” BC (ER+and/or PR+, HER2−),
“luminal B” BC (ER+and/or PR+, HER2+),
“triple-negative” BC (ER−, PR−, HER2−),
“HER2+/ER−” BC (ER−, PR−, and HER2+), or
“unclassified BC” (ER−, PR−, HER2−, cytokeratin 5/6−, and HER1−).

In the method of the invention, the level of a miR gene product in the urine sample, relative to the level of the respective miR gene product in a control sample, is typically increased by at least 10% or at least 25% or at least 50% or at least 100%, to be indicative of breast cancer.

The level of a miR gene product in the urine sample, relative to the level of the respective miR gene product in a control sample, is usually decreased by at least 10% or at least 25% or at least 50%, to be indicative of breast cancer.

In the method of the invention, the level of miR-660 gene product, and/or let7-i gene product in the urine sample from the subject, relative to the control level of the corresponding miR gene product, may be decreased by at least 25% to be indicative of breast cancer, and/or the level of miR-424 gene product, in the urine sample from the subject, relative to the control level of miR-424 gene product, may be increased by at least 50% to be indicative of breast cancer.

In another embodiment, the level of miR-660 gene product, and/or let7-i gene product in the urine sample from the subject, relative to the control level of the corresponding miR gene product, may be decreased by at least 50% to be indicative of breast cancer, and/or the level of miR-424 gene product, in the urine sample from the subject, relative to the control level of miR-424 gene product, may be increased by at least 100% to be indicative of breast cancer.

In another embodiment, the level of miR-660 gene product, and/or let7-i gene product in the urine sample from the subject, relative to the control level of the corresponding miR gene product, may be decreased by at least 75% to be indicative of breast cancer, and/or the level of miR-424 gene product, in the urine sample from the subject, relative to the control level of miR-424 gene product, may be increased by at least 200% to be indicative of breast cancer.

The subject may be diagnosed as having breast cancer if
the level of miR-424 gene product in the urine sample from the subject is greater than 110%, greater than 125%, greater than 150%, or greater than 200% of the control level of miR-424 gene product (e.g. the level in the control sample);
the level of miR-660 gene product in the urine sample from the subject is less than 90%, less than 75%, or less than 50% of the control level of miR-660 gene product (e.g. the level in the control sample);
the level of let7-i gene product in the urine sample from the subject is less than 90%, less than 75%, or less than 50% of the control level of let7-i gene product (e.g. the level in the control sample);
the level of miR-423 gene product in the urine sample from the subject is less than 90%, less than 75%, or less than 50% of the control level of miR-423 gene product (e.g. the level in the control sample);
the level of miR-125b gene product in the urine sample from the subject is greater than 110%, greater than 125%, greater than 150%, or greater than 200% of the control level of miR-125b gene product (e.g. the level in the control sample); and/or
the level of let7-d gene product in the urine sample from the subject is greater than 110%, greater than 125%, greater than 150%, or greater than 200% of the control level of let7-d gene product (e.g. the level in the control sample).

In a particular embodiment, the subject is diagnosed as having breast cancer if
the level of miR-424 gene product in the urine sample from the subject is greater than 110%, greater than 125%, greater than 150%, or greater than 200% of the control level of miR-424 gene product (e.g. the level in the control sample);
the level of miR-660 gene product in the urine sample from the subject is less than 90%, less than 75%, or less than 50% of the control level of miR-660 gene product (e.g. the level in the control sample); and
the level of let7-i gene product in the urine sample from the subject is less than 90%, less than 75%, or less than 50% of the control level of let7-i gene product (e.g. the level in the control sample);

and optionally if, in addition,
the level of miR-423 gene product in the urine sample from the subject is less than 90%, less than 75%, or less than 50% of the control level of miR-423 gene product (e.g. the level in the control sample);
the level of miR-125b gene product in the urine sample from the subject is greater than 110%, greater than 125%, greater than 150%, or greater than 200% of the control level of miR-125b gene product (e.g. the level in the control sample); or
the level of let7-d gene product in the urine sample from the subject is greater than 110%, greater than 125%, greater than 150%, or greater than 200% of the control level of let7-d gene product (e.g. the level in the control sample).

### Kits:

In another aspect, the invention pertains to a diagnostic kit, or a microarray, comprising an oligonucleotide capable of hybridizing to a nucleic acid consisting of SEQ ID NO:11, an oligonucleotide capable of hybridizing to a nucleic acid consisting of SEQ ID NO:12, and an oligonucleotide capable of hybridizing to a nucleic acid consisting of SEQ ID NO:17. The kit preferably further comprises an oligonucleotide capable of hybridizing to a nucleic acid consisting of SEQ ID NO:10, or an oligonucleotide capable of hybridizing to a nucleic acid consisting of SEQ ID NO:7, or an oligonucleotide capable of hybridizing to a nucleic acid consisting of SEQ ID NO:14.

As used herein, “capable of hybridizing” preferably refers to high stringency hybridization conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. High stringency conditions would include, for example, low salt and/or high temperature conditions, such as provided by 0.02-0.1 M NaCl, at temperatures of 50-70° C. (e.g. 0.05 M NaCl at 60° C.).

In yet another aspect, the invention relates to the use of an oligonucleotide capable of hybridizing to a nucleic acid consisting of SEQ ID NO:11, an oligonucleotide capable of hybridizing to a nucleic acid consisting of SEQ ID NO:12, and/or an oligonucleotide capable of hybridizing to a nucleic acid consisting of SEQ ID NO:17 for the diagnosis of breast cancer, said use comprising contacting said oligonucleotide(s) with a urine sample. The use preferably further comprises contacting an oligonucleotide capable of hybridizing to a nucleic acid consisting of SEQ ID NO:10, an oligonucleotide capable of hybridizing to a nucleic acid consisting of SEQ ID NO:7, or an oligonucleotide capable of hybridizing to a nucleic acid consisting of SEQ ID NO:14 with said urine sample. The oligonucleotides may be immobilized on a microarray.

In particular embodiments of the kit or use described above, the oligonucleotide capable of hybridizing to a nucleic acid consisting of SEQ ID NO:11 is perfectly complementary to SEQ ID NO:11, the oligonucleotide capable of hybridizing to a nucleic acid consisting of SEQ ID NO:12 is perfectly complementary to SEQ ID NO:12, and/or the oligonucleotide capable of hybridizing to a nucleic acid consisting of SEQ ID NO:17 is perfectly complementary to SEQ ID NO:17. Additionally, the oligonucleotide capable of hybridizing to a nucleic acid consisting of SEQ ID NO:10 may be perfectly complementary to SEQ ID NO:10. Additionally, the oligonucleotide capable of hybridizing to a nucleic acid consisting of SEQ ID NO:7 may be perfectly complementary to SEQ ID NO:7. Additionally, the oligonucleotide capable of hybridizing to a nucleic acid consisting of SEQ ID NO:14 may be perfectly complementary to SEQ ID NO:14. As used herein the term “complementary” refers to the reverse complement, unless indicated otherwise.

In other embodiments, the kit contains a pair of primers for amplifying a miR described herein. The kit may comprise a primer pair capable of amplifying miR-424, a primer pair capable of amplifying miR-660, and/or a primer pair capable of amplifying let7-i. The kit may further comprise a primer pair capable of amplifying miR-423, a primer pair capable of amplifying miR-125b, and/or a primer pair capable of amplifying let7-d. The kit may further comprise a primer pair capable of amplifying miR-17, a primer pair capable of amplifying let7-f, a primer pair capable of amplifying miR-222 and/or a primer pair capable of amplifying miR-194.

The kit may further comprise a universal primer for the reverse transcription reaction.

This diagnostic kit can be used for breast screening, e.g. by identifying changes in urine miRNA levels in breast cancer patients compared to normal cancer-free individuals, in a control group. The kit may further be used for the prognosis and/or prediction of outcome, e.g. by identifying differences between patients with early or late stage cancers, as well as stratifying patients into molecular subtypes. This information can then aid in strategic planning of an individual patients therapeutic regimen. Further, the kit may be used for the monitoring of response to treatments, e.g. through serial urinary miRNA measurements; particularly in the neoadjuvant chemotherapy and metastatic disease settings.

The methods, uses and kits of the invention have been described above in connection with breast cancer. The invention is also applicable for the diagnosis, detection and/or screening of other cancer types, e.g. of breast carcinomas, lung carcinomas, gastric carcinomas, esophageal carcinomas, colorectal carcinomas, liver carcinomas, ovarian carcinomas, thecomas, arrhenoblastomas, cervical carcinomas, endometrial carcinoma, endometrial hyperplasia, endometriosis, fibrosarcomas, choriocarcinoma, head and neck cancer, nasopharyngeal carcinoma, laryngeal carcinomas, hepatoblastoma, Kaposi's sarcoma, melanoma, skin carcinomas, hemangioma, cavernous hemangioma, hemangioblastoma, pancreas carcinomas, retinoblastoma, astrocytoma, glioblastoma, Schwannoma, oligodendroglioma, medulloblastoma, neuroblastomas, rhabdomyosarcoma, osteogenic sarcoma, leiomyosarcomas, urinary tract carcinomas, thyroid carcinomas, Wilm's tumor, renal cell carcinoma, prostate carcinoma, abnormal vascular proliferation associated with phakomatoses, edema (such as that associated with brain tumors), and/or Meigs' syndrome. The embodiments described above in connection with BC apply to theses other cancer types mutatis mutandis.

Another aspect of the present invention is a method for enriching or isolating extracellular vesicles, in particular exosomes, from a body fluid sample. The body fluid can be any body fluid, including, but not limited to, blood, plasma, urine, ascites, amniotic liquor, and saliva.

The method comprises filtrating the body fluid sample through a filter membrane which has protein binding properties. The filter membrane is preferably a polyamide membrane, e.g. a

Nylon membrane. The pore size is typically from about 0.1 μm to about 1 μm, preferably from about 0.15 μm to about 500 μm, more preferably from about 0.18 μm to about 0.25 μm, e.g. about 0.22 μm.

In certain embodiments the method further comprises, prior to the filtration through the protein-binding membrane, a pre-filtration through a membrane which does not have protein-binding properties. The pre-filtration may serve to separate larger EVs, such as microvesicles, from exosomes which have a smaller size. Accordingly, the pore size of the pre-filtration membrane is typically from about 0.18 μm to about 0.25 μm, preferably from about 0.19 μm to about 0.21 μm, e.g. about 0.2 μm. The pre-filtration membrane may be made of a cellulosic material such as cellulose acetate.

Initially the body fluid sample may be centrifuged to remove cell debris.

After filtration through the protein-binding membrane the exosomes are bound to the membrane. The method of the invention further comprises recovering the exosomes from the membrane. This is typically achieved by contacting the membrane with a suitable solution such as a lysis buffer. Suitable buffers may include a buffer substance to maintain the pH of the lysis buffer in a range from about 6.5 to 8, or 7 to 7.5, e.g. about 7.2. The lysis buffer may contain a surfactant or detergent. Suitable surfactancts include sodium lauryl sulfate and Triton X100.

The method for enriching or isolating exosomes or extracellular vesicles can be used in the method of diagnosing cancer described herein, e.g. for providing the urine sample.

The following examples illustrate the invention and should not be understood as limiting the invention to the exemplified embodiments.

### EXAMPLES

### Example 1

### 1. Methods

### 1.1 Cohort

The case-control cohort comprised of 69 untreated patients, newly diagnosed with primary BC in the adjuvant setting and 40 healthy female controls at the Department of Obstetrics and Gynecology, University Medical Center Freiburg. Control's health status was confirmed by medical examination performed by an experienced clinical physician including breast and regional lymph nodes to exclude potential BC and any history of other (malignant) disease or current inflammation. Furthermore healthy controls underwent breast and axillary ultrasound and mammography. By staging procedures according to the current national guidelines, all BC patients were tested to preclude cases of advanced stages with distant metastasis.

The according investigation protocols (36/12 and 386/16) were approved by the institutional ethical review board of the University of Freiburg. All patients and healthy controls involved provided written informed consent. Clinical cohort characteristics are summarized in Table 2.

TABLE 2
Cohort characteristics of breast cancer (BC) patients and healthy controls

| | BC patients | healthy controls | p value |
|---|---|---|---|
| N | 69 | 40 | |
| median age, y | 58 (30-79) | 45 (20-72) | 0. |
| Histology | | | |
| invasive ductal | 49 | | |
| invasive lobular | 5 | | |
| Tumor stage | | | |
| pT1a | 2 | | |
| pT1b | 5 | | |
| pT1c | 39 | | |
| pT2 | 17 | | |
| pT3 | 2 | | |
| Nodal status | | | |
| pN0 | 53 | | |
| pN1 | 11 | | |
| pN2 | 1 | | |
| Grading | | | |
| G1 | 10 | | |
| G2 | 43 | | |
| G3 | 8 | | |
| Hormone receptor | | | |
| status | | | |
| ER positive | 53 | | |
| PR positive | 50 | | |
| HER2neu status | | | |
| positive | 4 | | |

1.2 miRNA Specimen

This biomarker identification study with diagnostic applicability in respect of breast cancer detection based on the expression analysis of urinary circulating miRNA types. The chosen set of thirteen miRNA specimen is characterized by the combined features of a proven functional relation to breast cancer and a robust and reliable detectability in human urine samples. Due to normalization purposes in relative quantification of miRNA expression levels two miRNA specimen with housekeeping characteristics (miR16, -26b) could be identified in a previous study [12]. Definite information on miRNA types including target sequences are listed in Table 2.

TABLE 2
Information on miRNA names and sequences utilized
in expression analysis.

| | | SEQ |
|---|---|---|
| miRNA | miRNA sequence | ID NO: |
| cel-miR39-3p^{#} | UCACCGGGUGUAAAUCAGCUUG | 1 |
| ath-miR159a^{#} | UUUGGAUUGAAGGGAGCUCUA | 2 |
| hsa-miR16-5p | UAGCAGCACGUAAAUAUUGGCG | 3 |
| hsa-miR17-5p | CAAAGUGCUUACAGUGCAGGUAG | 4 |
| hsa-miR26b-5p | UUCAAGUAAUUCAGGAUAGGU | 5 |
| hsa-miR107-5p | AGCAGCAUUGUACAGGGCUAUCA | 6 |
| hsa-miR125b-5p | UCCCUGAGACCCUAACUUGUGA | 7 |
| hsa-miR194-5p | UGUAACAGCAACUCCAUGUGGA | 8 |
| hsa-miR222-3p | AGCUACAUCUGGCUACUGGGU | 9 |
| hsa-miR423-5p | UGAGGGGCAGAGAGCGAGACUUU | 10 |
| hsa-miR424-5p | CAGCAGCAAUUCAUGUUUUGAA | 11 |
| hsa-miR660-5p | UACCCAUUGCAUAUCGGAGUUG | 12 |
| hsa-let7a-5p | UGAGGUAGUAGGUUGUAUAGUU | 13 |
| hsa-let7d-5p | AGAGGUAGUAGGUUGCAUAGUU | 14 |
| hsa-let7e-5p | UGAGGUAGGAGGUUGUAUAGUU | 15 |
| hsa-let7f-5p | UGAGGUAGUAGAUUGUAUAGUU | 16 |
| hsa-let7i-5p | UGAGGUAGUAGUUUGUGCUGUU | 17 |

^{#}exogenous, synthetic spike-in control RNA specimen

### 1.3 Sampling and Storage

Native spontaneous urine samples were collected in 100 ml sterile urine sampling cups

(Sarstedt, Germany) and stored in 10 ml aliquots (Urin Monovette, Sarstedt, Germany) at −80° C. until further processing.

### 1.4 Sample Preparation, RNA Isolation and Reverse Transcription

Cryopreserved native urine specimen were thawed at room temperature and centrifuged (4000 rpm; 15min; 4° C.) to remove potential cell debris residues or other solid precipitates from pure urine supernatant.

For isolation of miRNA-loaded exosomes/microvesicles from urine samples, 10 ml centrifuged urine were transferred to a 10 ml syringe (#4617207V; BRAUN, Melsungen, Germany) with a screwed on 0.22 μm nylon syringe filter (#02542904; Perkin Elmer, Waltham, USA). With piston inserted, the sample was filtered dropwise through the screwed-on filter. Following filtration, the filters were washed with 5 ml DPBS buffer (#14190185; ThermoFisher Scientific, Karlsruhe, Germany) using a fresh 5 ml syringe (#4617053V; BRAUN, Melsungen, Germany) to remove sample residues that possibly interfere with downstream applications. After washing, total RNA isolation was performed utilizing the Norgen Total RNA Purification Kit (#17200, NORGEN Biotek Corp., Thorold, Canada). According to manufacturer's protocol [58] (Appendix B: ‘Total RNA Purification from Plasma or Serum’) 600 μl lysis buffer were extruded through filter membrane. Purified RNA was eluted in 35 μl elution buffer. Further processing of RNA specimen was defined to a standard volume of 2.5 μl.

Reverse transcription (RT) of miRNAs was realized by utilizing 2.5 μl of RNA sample, 5 μl of 5× RT-Buffer, 0.1 mM of ATP, 0.5 μM RT-primer, 0.1 mM of each deoxynucleotide (dATP, dCTP, dGTP, dTTP), 25 units of Maxima Reverse Transcriptase (Thermo) and 1 unit of poly(A) polymerase (New England Biolabs GmbH, Frankfurt, Germany) in a final volume of 25 μl Reaction incubation was set to 30 min at 42° C. followed by enzyme inactivation at 85° C. for 10 min.

Primer design for RT reaction was geared to ThiRprimer software tool by Busk [59]. RT Primer information is listed in Table 3. cDNA samples were diluted in H2O to a final volume of 200 μl and stored at −20° C. until further processing.

TABLE 3
Information on primers utilized in expression analysis of miRNA types.

| miRNA | sense primer | antisense primer |
|---|---|---|
| cel-miR39-3p^{#} | GTCACCGGGTGTAAATCAG | GGTCCAGTTTTTTTTTTTTTTTCAAG |
| | (SEQ ID NO: 18) | (SEQ ID NO: 19) |
| ath-miR159a^{#} | GCGCAGTTTGGATTGAAG | AGGTCCAGTTTTTTTTTTTTTTTAGAG |
| | (SEQ ID NO: 20) | (SEQ ID NO: 21) |
| hsa-miR16-5p | CGCAGTAGCAGCACGTA | CAGTTTTTTTTTTTTTTTCGCCAA |
| | (SEQ ID NO: 22) | (SEQ ID NO: 23) |
| hsa-miR17-5p | GCAAAGTGCTTACAGTGCAG | GGTCCAGTTTTTTTTTTTTTTTCTAC |
| | (SEQ ID NO: 24) | (SEQ ID NO: 25) |
| hsa-miR26b-5p | CGCAGTTCAAGTAATTCAGGAT | GGTCCAGTTTTTTTTTTTTTTTACCT |
| | (SEQ ID NO: 26) | (SEQ ID NO: 27) |
| hsa-miR107-5p | GCAGAGCAGCATTGTACAG | GGTCCAGTTTTTTTTTTTTTTTGATAG |
| | (SEQ ID NO: 28) | (SEQ ID NO: 29) |
| hsa-miR125b-5p | GCAGTCCCTGAGACCCT | CCAGTTTTTTTTTTTTTTTCACAAGT |
| | (SEQ ID NO: 30) | (SEQ ID NO: 31) |
| hsa-miR194-5p | CAGTGTAACAGCAACTCCA | TCCAGTTTTTTTTTTTTTTTCCACAT |
| | (SEQ ID NO: 32) | (SEQ ID NO: 33) |
| hsa-miR222-3p | GCAGAGCTACATCTGGCT | CCAGTTTTTTTTTTTTTTTACCCAGT |
| | (SEQ ID NO: 34) | (SEQ ID NO: 35) |
| hsa-miR423-5p | CAGTGAGGGGCAGAGAG | GGTCCAGTTTTTTTTTTTTTTTAAAGTC |
| | (SEQ ID NO: 36) | (SEQ ID NO: 37) |
| hsa-miR424-5p | AGCAGCAGCAATTCATGT | AGGTCCAGTTTTTTTTTTTTTTTCAA |
| | (SEQ ID NO: 38) | (SEQ ID NO: 39) |
| hsa-miR660-5p | AGTACCCATTGCATATCGGA | GGTCCAGTTTTTTTTTTTTTTTCAAC |
| | (SEQ ID NO: 40) | (SEQ ID NO: 41) |
| hsa-let7a-5p | GCAGTGAGGTAGTAGGTTG | GGTCCAGTTTTTTTTTTTTTTTAACTATAC |
| | (SEQ ID NO: 42) | (SEQ ID NO: 43) |
| hsa-let7d-5p | CGCAGAGAGGTAGTAGGTTG | GGTCCAGTTTTTTTTTTTTTTTAACTATG |
| | (SEQ ID NO: 44) | (SEQ ID NO: 45) |
| hsa-let7e-5p | GCAGTGAGGTAGGAGGTTG | GGTCCAGTTTTTTTTTTTTTTTAACTATAC |
| | (SEQ ID NO: 46) | (SEQ ID NO: 47) |
| hsa-let7f-5p | CGCAGTGAGGTAGTAGATTG | CAGGTCCAGTTTTTTTTTTTTTTTAAC |
| | (SEQ ID NO: 48) | (SEQ ID NO: 49) |
| hsa-let7i-5p | GCAGTGAGGTAGTAGTTTGTG | GGTCCAGTTTTTTTTTTTTTTTAACAG |
| | (SEQ ID NO: 50) | (SEQ ID NO: 51) |
| miRNA RT* | CAGGTCCAGTTTTTTTTTTTTTTTVN | |
| | (SEQ ID NO: 52) | |

^{#}exogenous, synthetic spike-in control RNA specimen; *universal primer for reverse transcription (RT) reaction.

1.5 qPCR-Based miRNA Quantification

Quantitative determination of miRNA expression levels was realized by real-time PCR on LightCycler® 480 (Roche, Mannheim, Germany). PCR reaction set up: 1 μl cDNA, in-house qPCR buffer (containing TRIS pH8.1, dATP, dCTP, dGTP, dTTP, magnesium, potassium ammonium, SYBRGreen (Jena Bioscience, Jena, Germany), enhancers) 0.25U HotStart Taq Polymerase (Jena Bioscience) in a total volume of 10 μl.

qPCR primers were designed via ThiRprimer software [59] and are listed in Table 3.

PCR program comprised of initial denaturation (95° C.; 2min); 40 cycles: denaturation (95° C.; 5 sec)/annealing/extension (60° C.; 30sec); melting curve.

Relative quantification of miRNA expression levels was performed in a duplicate analysis based on ΔCt method normalized on corresponding mean expression values of housekeeping miRNAs miR-16 and -26b. Validation of housekeeping miRNAs applicable for the setting of urinary miRNA analysis was proven in our previous study [12] and is methodically described by Marabita et al. [60].

### 1.6 Technical Processing Normalization via Spike-In Control RNAs

Monitoring of processing efficiency in regard to RNA extraction, complementary DNA synthesis (reverse transcription) and PCR amplification was realized by addition of ‘spike-in’ control RNA specimen of defined concentration to the RNA lysis buffer as recommended by Marabita et al. [60]. Thereto, 5fM two exogenous synthetic RNA types (Caenorhabditis elegans: cel-miR-39 and Arabidopsis thaliana: ath-miR-159; Biomers.net GmbH, Ulm, Germany; sequences in Table 3) were added to RNA lysis buffer (Norgen).

Technical normalization of expression data obtained by qPCR was performed based on threshold points (Cₜ) with relative quantities (RQs) defined as those Cₜ values scaled to the geometric mean of external reference spike-in RNAs and conversion to a linear scale RQ=2^{-ΔCt} with ΔCₜ=C_{t miRNA}−C_{t spike geometric mean}.

Normalization factor (NF) was calculated as geometric mean of the selected normalizers (housekeeper miRNA specimen (miR16, −26b) for each sample (j). Thus, the NF is applied to extract the normalized relative quantities (NRQ) for each miRNA i and j sample: NRQij=RQij/NFj [60].

### 1.7 Statistical Analyses

Biomarker assessment focused on the extraction of the diagnostic value of all thirteen analyzed miRNA types and combinations of them in regard to urine-based breast cancer detection. To this aim we used several statistical approaches, described as follows.
1.Variable selection frequencies with cross validation: In each of 10,000 repetitions, we randomly selected a subsample of size ⅔ of the data (the training sample), fitted a logistic regression model using forward selection and determined the inclusion frequency of each variable. We then chose the 7 variables with the highest inclusion frequency, fitted a model to the full data set and reduced this model further by forward selection, finally choosing within all convergent models the one that minimized the Akaike information criterion (AIC).
2. Variable selection frequencies with bootstrap: We drew 10,000 bootstrap samples (with repetition). In each bootstrap sample, we performed modelling and variable selection as in the first approach.
3. ROC analysis/cross validation: Similar to approach 1, but now we applied the model that was fitted in the training sample to the (remaining) test sample, with fixed coefficients (100 repetitions). We constructed the receiver operating characteristic (ROC) curves and used the area under the curve (AUC) as a measure of goodness of fit.
4. Variable selection frequencies with boosting: Boosting is a stepwise and regularized procedure for fitting generalized linear models. Starting with setting all regression coefficients to zero, in each step one coefficient is updated, such that the model fit improves most. The number of steps is determined by cross-validation. As many coefficients will never be updated, boosting provides a sparse model. The result was visualized by showing the coefficient paths. Boosting was performed using the R package GAMBoost [61].
5.All subsets regression: All possible 2¹²=4096 models were fitted and compared based on the AIC. We then restricted the list to convergent models with at most 4 variables.

All analyses were performed using the open statistical software environment R [62].

### 2. Results

The complete panel of all thirteen circulating miRNA specimen could be reliably detected in all urine specimen of the test cohort. Sample-dependent variations in expression levels of miRNAs due to variable urine parameters (e.g. dilution, concentration of nucleic acids) could be equilibrated by normalization against the set of two stably expressed housekeeping miRNAs 16 and 26b.

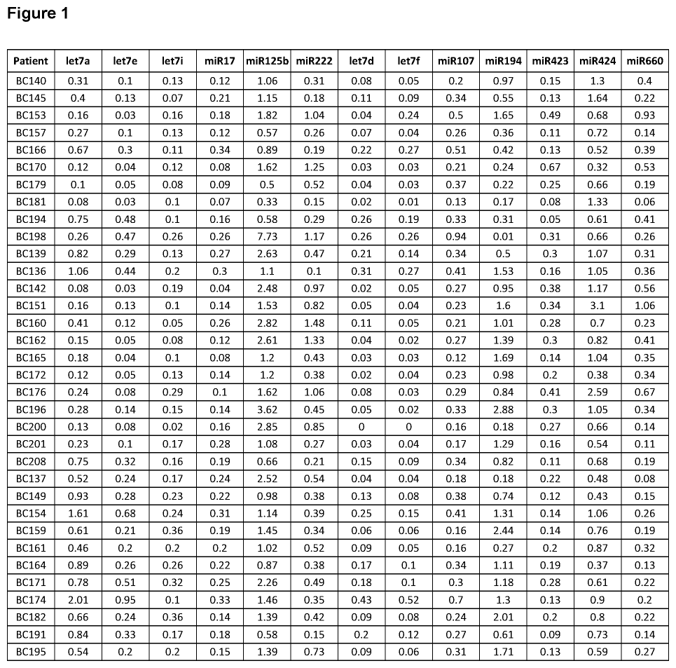

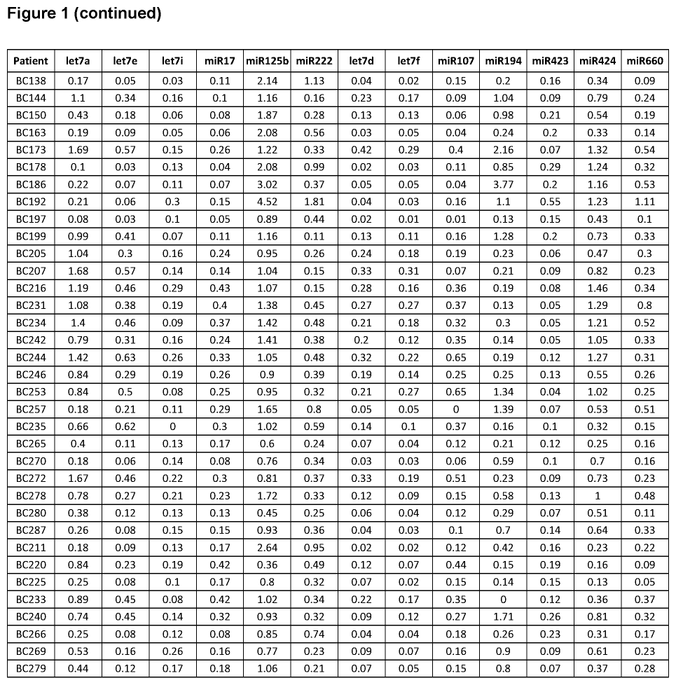

Based on qPCR-derived miRNA expression levels converted to normalized relative quantities (NRQ; FIG. 1 and FIG. 2), the assessment of miRNA type-specific diagnostic power in regard to discrimination purposes of breast cancer cases against healthy controls was evaluated by employing several statistical methods (see Methods section). Thereby the applicability and validity of the methods were compared. These methods were considered in both, a singular fashion each and in a combined manner. The summarized information output of the utilized statistical analyses is described in the following (1)-(5).

(1) Variable Selection Frequencies with Cross Validation

The empirical cumulative distribution functions of the regression coefficients over all 10,000 repetitions resulted in the following ranking scheme in miRNA type frequencies (inclusion frequency, relating to all selected models, in brackets; up=upregulated expression, down=downregulated expression).
1. miR-424 (95.98%; up)
2. miR-423 (72.3%; down)
3. miR-660 (68.7%; down)
4. let7-1 (59.97%; down)
5. miR-17 (15.96%; up)
6. let7-f (14.65%; down)
7. miR-222 (9.82%; up)
8. miR-194 (8,59%; up)
9. miR-125b (3.57%; up)
10. let7-d (3.25%; down)

The forward-selection based on these ten variables (miRNA types) led to a model including the first five variables (miR-424, miR-423, miR-660, let7-i, miR-17). Since models with more than four variables (miRNA types) failed to converge, these were excluded from further statistical assessment. Applying these restrictions, this statistical approach provided the model: miR-424+miR-423+miR-660+let7-i.

(2) Variable Selection Frequencies with Bootstrap

The empirical cumulative distribution functions of the regression coefficients over all 10,000 repetitions resulted in the following ranking of the most frequently chosen miRNA types was found.
1. miR-424 (94.81%; up)
2. miR-660 (64.23%; down)
3. miR-423 (58.96%; down)
4. let7-i (58.96%; down)
5. let7-f (20.32%; down)
6. miR-17 (12.46%; up)
7. miR-222 (10.79%, up)
8. miR-194 (9.44%; up)
9. miR-125b (5.82%; up)
10. let7-d (3.57%; up)

Forward-selection led to a model with the five miRNA types let7-i, let7-f, miR-423, miR-424, miR-660. Again, the missing convergence of models with more than four variables led to the reduction to the following miRNA type combination, which is identical to the result of the first statistical approach: miR-424+miR-423+miR-660+let7-i.

### (3) ROC Analysis/Cross Validation

The previously preferred model with four variables miR-424, miR-423, miR-660, let7-i (1, 2)) was selected in 18 of 100 repetitions. In all these repetitions the AUC was 1 both for the training sample and for the test sample, if the same model was newly fitted. When we applied the fitted model with fixed coefficients to the test sample, AUC values ranged between 0.905 and 1 with a median value of 0.995.

There was a number of alternative models with at most four miRNA types with promising AUC values. The most frequently selected combination of five miRNA types was again miR-424, miR-423, miR-660, let7-i, and miR-17.

(4) Variable Selection Frequencies with Boosting

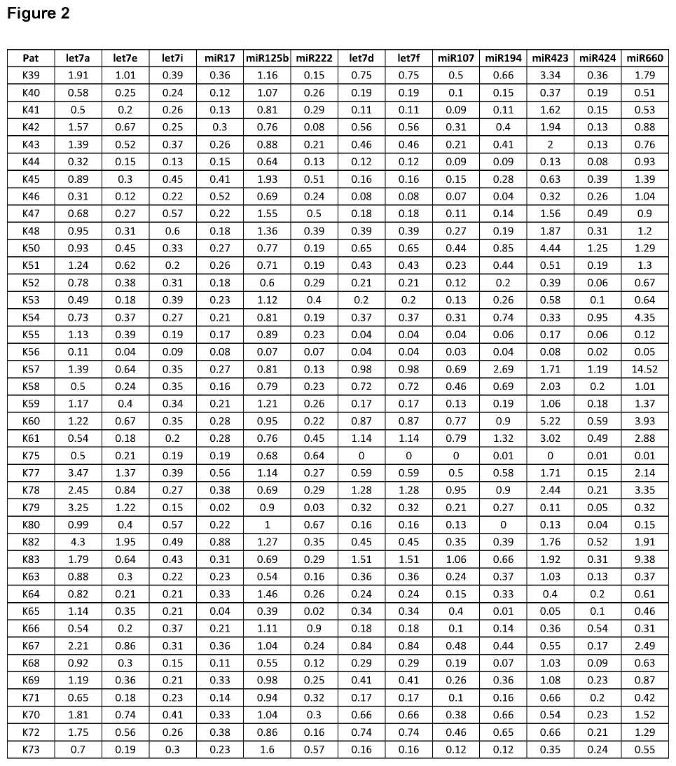

After 1500 boosting steps, seven miRNA types had been selected (including those obtained by the earlier approaches). These were miR-424 (up), miR-423 (down), miR-660 (down), let-7i (down), miR-194 (up), let7-a (down), miR-125b (up), see FIG. 3.

### (5) All Subsets Regression

All 4096 possible candidate models were fitted. The restriction to convergent models with at most four variables provided the following superior models:
1. miR-424+miR-660+let7-i+miR-125b (AIC=15.0)
2. miR-424+mi-R423+miR-660+let7-i (AIC=16.3)
3. miR-424+miR-660+let7-i+let7-d (AIC=17.2)
4. miR-424+miR-660+let7-i (AIC=19.2)

The first model characterized by the lowest AIC (AIC=15.0) occurred only here, not in the previous approaches. The second model contains the four miRNA types (miR-424+miR-423+miR-660+let7-i) with the highest inclusion frequency in approaches (1) and (2). The second model occurred also in approach (3).

Note that all subsets regression works without cross validation, as all models are fitted to the full data set. Thus, this regression method lacks information as regards the predictive performance of the models.

Summing up, a panel of four miRNA types, comprising miR-424, miR-423, miR-660, and let7-i could be elected as highly specific combinatory biomarker tool in discrimination of breast cancer patients vs. healthy controls based on urine specimen.

### Abbreviations

AIC Akaike information criterion
AUC area under the curve
miRNA/miR micro ribonucleic acid
NF normalization factor
NGS next generation sequencing
NRQ normalized relative quantities
PET positron-emission tomography
qPCR quantitative polymerase chain reaction
ROC receiver operating characteristic
SNP single nucleotide polymorphism

### REFERENCES

1. Disease, G.B.D., I. Injury, and C. Prevalence, Global, regional, and national incidence, prevalence, and years lived with disability for 328 diseases and injuries for 195 countries, 1990-2016: a systematic analysis for the Global Burden of Disease Study 2016. Lancet, 2017. 390(10100): p. 1211-1259.

2. Sharma, R., Breast cancer incidence, mortality and mortality-to-incidence ratio (MIR) are associated with human development, 1990-2016: evidence from Global Burden of Disease Study 2016. Breast Cancer, 2019.

3. Momenimovahed, Z. and H. Salehiniya, Epidemiological characteristics of and risk factors for breast cancer in the world. Breast Cancer (Dove Med Press), 2019. 11: p. 151-164.

4. Oeffinger, K. C., et al., Breast Cancer Screening for Women at Average Risk: 2015 Guideline Update From the American Cancer Society. JAMA, 2015. 314(15): p. 1599-614.

5. Tabar, L., et al., Insights from the breast cancer screening trials: how screening affects the natural history of breast cancer and implications for evaluating service screening programs. Breast J, 2015. 21(1): p. 13-20.

6. Wockel, A., et al., Interdisciplinary Screening, Diagnosis, Therapy and Follow-up of Breast Cancer. Guideline of the DGGG and the DKG (S3-Level, AWMF Registry Number 032/045OL, December 2017)—Part 1 with Recommendations for the Screening, Diagnosis and Therapy of Breast Cancer. Geburtshilfe Frauenheilkd, 2018. 78(10): p. 927-948.

7. Sankaranarayanan, R., et al., Cancer survival in Africa, Asia, and Central America: a population-based study. Lancet Oncol, 2010. 11(2): p. 165-73.

8. Kolb, T. M., J. Lichy, and J. H. Newhouse, Comparison of the performance of screening mammography, physical examination, and breast US and evaluation of factors that influence them: an analysis of 27,825 patient evaluations. Radiology, 2002. 225(1): p. 165-75.

9. Wang, L., Early Diagnosis of Breast Cancer. Sensors (Basel), 2017. 17(7).

10. Arneth, B., Update on the types and usage of liquid biopsies in the clinical setting: a systematic review. BMC Cancer, 2018. 18(1): p. 527.

11. Salehi, M. and M. Sharifi, Exosomal miRNAs as novel cancer biomarkers: Challenges and opportunities. J Cell Physiol, 2018. 233(9): p. 6370-6380.

12. Erbes, T., et al., Feasibility of urinary microRNA detection in breast cancer patients and its potential as an innovative non-invasive biomarker. BMC Cancer, 2015. 15: p. 193.

13. Balacescu, O., et al., Urinary microRNAs for prostate cancer diagnosis, prognosis, and treatment response: are we there yet? Wiley Interdiscip Rev RNA, 2017. 8(6).

14. Gasparri, M. L., et al., Beyond circulating microRNA biomarkers: Urinary microRNAs in ovarian and breast cancer. Tumour Biol, 2017. 39(5): p. 1010428317695525.

15. Grayson, K., et al., Urine Biomarkers for the Early Detection of Ovarian Cancer—Are We There Yet? Biomark Cancer, 2019. 11: p. 1179299X19830977.

16. Franzen, C. A., et al., Urinary Exosomes: The Potential for Biomarker Utility, Intercellular Signaling and Therapeutics in Urological Malignancy. J Urol, 2016. 195(5): p. 1331-1339.

17. Balzeau, J., et al., The LIN28/let-7 Pathway in Cancer. Front Genet, 2017. 8: p. 31.

18. Dufresne, S., et al., A Review of Physical Activity and Circulating miRNA Expression: Implications in Cancer Risk and Progression. Cancer Epidemiol Biomarkers Prey, 2018. 27(1): p. 11-24.

19. Elghoroury, E. A., et al., Evaluation of miRNA-21 and miRNA Let-7 as Prognostic Markers in Patients With Breast Cancer. Clin Breast Cancer, 2018. 18(4): p. e721-e726.

20. Masood, N., et al., Entangling Relation of Micro RNA-let7, miRNA-200 and miRNA-125 with Various Cancers. Pathol Oncol Res, 2017. 23(4): p. 707-715.

21. Sen, C. K., et al., Micromanaging vascular biology: tiny microRNAs play big band. J Vasc Res, 2009. 46(6): p. 527-40.

22. Thammaiah, C. K. and S. Jayaram, Role of let-7 family microRNA in breast cancer. Noncoding RNA Res, 2016. 1(1): p. 77-82.

23. de Anda-Jauregui, G., et al., Nonredundant, Highly Connected MicroRNAs Control Functionality in Breast Cancer Networks. Int J Genomics, 2018. 2018: p. 9585383.

24. Lehmann, T. P., et al., Relative levels of let-7a, miR-17, miR-27b, miR-125a, miR-125b and miR-206 as potential molecular markers to evaluate grade, receptor status and molecular type in breast cancer. Mol Med Rep, 2015. 12(3): p. 4692-4702.

25. Sun, H., et al., Let7 miRNAs sensitize breast cancer stem cells to radiationinduced repression through inhibition of the cyclin D1/Akt1/Wnt1 signaling pathway. Mol Med Rep, 2016. 14(4): p. 3285-92.

26. Bobbili, M. R., et al., OncomiR-17-5p: alarm signal in cancer? Oncotarget, 2017. 8(41): p. 71206-71222.

27. Hesari, A., et al., Expression of circulating miR-17, miR-25, and miR-133 in breast cancer patients. J Cell Biochem, 2018.

28. Li, J., et al., miR-17-5p suppresses cell proliferation and invasion by targeting ETV1 in triple-negative breast cancer. BMC Cancer, 2017. 17(1): p. 745.

29. Swellam, M., et al., Role of some circulating MiRNAs on breast cancer diagnosis. Arch Physiol Biochem, 2018: p. 1-9.

30. Wang, Y., et al., MiR-17-5p may serve as a novel predictor for breast cancer recurrence. Cancer Biomark, 2018. 22(4): p. 721-726.

31. Feliciano, A., et al., miR-125b acts as a tumor suppressor in breast tumorigenesis via its novel direct targets ENPEP, CK2-alpha, CCNJ, and MEGF9. PLoS One, 2013. 8(10): p. e76247.

32. Ferracin, M., et al., miR-125b targets erythropoietin and its receptor and their expression correlates with metastatic potential and ERBB2/HER2 expression. Mol Cancer, 2013. 12(1): p. 130.

33. Luo, Y., et al., Elevated microRNA-125b levels predict a worse prognosis in HER2-positive breast cancer patients. Oncol Lett, 2017. 13(2): p. 867-874.

34. Mar-Aguilar, F., et al., Differential expression of miR-21, miR-125b and miR-191 in breast cancer tissue. Asia Pac J Clin Oncol, 2013. 9(1): p. 53-9.

35. Amini, S., et al., Expression Analysis of MicroRNA-222 in Breast Cancer. Clin Lab, 2018. 64(4): p. 491-496.

36. Liu, S., et al., miR-221/222 activate the Wnt/beta-catenin signaling to promote triple-negative breast cancer. J Mol Cell Biol, 2018. 10(4): p. 302-315.

37. Liang, Y. K., et al., MiR-221/222 promote epithelial-mesenchymal transition by targeting Notch3 in breast cancer cell lines. NPJ Breast Cancer, 2018. 4: p. 20.

38. Han, S. H., et al., MicroRNA-222 Expression as a Predictive Marker for Tumor Progression in Hormone Receptor-Positive Breast Cancer. J Breast Cancer, 2017. 20(1): p. 35-44.

39. Ding, J., et al., Exosome-mediated miR-222 transferring: An insight into NF-kappaB-mediated breast cancer metastasis. Exp Cell Res, 2018. 369(1): p. 129-138.

40. Gao, B., et al., MicroRNA-107 is downregulated and having tumor suppressive effect in breast cancer by negatively regulating brain-derived neurotrophic factor. J Gene Med, 2017. 19(12).

41. Petrovic, N., et al., MicroRNA in breast cancer: The association with BRCA1/2. Cancer Biomark, 2017. 19(2): p. 119-128.

42. Ai, H., et al., microRNAs-107 inhibited autophagy, proliferation, and migration of breast cancer cells by targeting HMGB1. J Cell Biochem, 2018.

43. Zhang, L., et al., MiR-107 down-regulates SIAH1 expression in human breast cancer cells and silencing of miR-107 inhibits tumor growth in a nude mouse model of triple-negative breast cancer. Mol Carcinog, 2016. 55(5): p. 768-77.

44. Li, X. Y., et al., MiRNA-107 inhibits proliferation and migration by targeting CDK8 in breast cancer. Int J Clin Exp Med, 2014. 7(1): p. 32-40.

45. Kleivi Sahlberg, K., et al., A serum microRNA signature predicts tumor relapse and survival in triple-negative breast cancer patients. Clin Cancer Res, 2015. 21(5): p. 1207-14.

46. Stuckrath, I., et al., Aberrant plasma levels of circulating miR-16, miR-107, miR-130a and miR-146a are associated with lymph node metastasis and receptor status of breast cancer patients. Oncotarget, 2015. 6(15): p. 13387-401.

47. Yang, F., Z. Xiao, and S. Zhang, Knockdown of miR-194-5p inhibits cell proliferation, migration and invasion in breast cancer by regulating the Wnt/beta-catenin signaling pathway. Int J Mol Med, 2018. 42(6): p. 3355-3363.

48. Huo, D., et al., Identification of a circulating microRNA signature to distinguish recurrence in breast cancer patients. Oncotarget, 2016. 7(34): p. 55231-55248.

49. Le, X. F., et al., Modulation of MicroRNA-194 and cell migration by HER2-targeting trastuzumab in breast cancer. PLoS One, 2012. 7(7): p. e41170.

50. Zhao, H., et al., Genetic analysis and preliminary function study of miR-423 in breast cancer. Tumour Biol, 2015. 36(6): p. 4763-71.

51. Smith, R. A., et al., A genetic variant located in miR-423 is associated with reduced breast cancer risk. Cancer Genomics Proteomics, 2012. 9(3): p. 115-8.

52. Murria Estal, R., et al., MicroRNA signatures in hereditary breast cancer. Breast Cancer Res Treat, 2013. 142(1): p. 19-30.

53. Wang, J., et al., miR-424-5p regulates cell proliferation, migration and invasion by targeting doublecortin-like kinase 1 in basal-like breast cancer. Biomed Pharmacother, 2018. 102: p. 147-152.

54. Rodriguez-Barrueco, R., et al., miR-424(322)/503 is a breast cancer tumor suppressor whose loss promotes resistance to chemotherapy. Genes Dev, 2017. 31(6): p. 553-566.

55. Zhang, L., et al., A circulating miRNA signature as a diagnostic biomarker for non-invasive early detection of breast cancer. Breast Cancer Res Treat, 2015. 154(2): p. 423-34.

56. Shen, Y., et al., Inhibition of miR-660-5p expression suppresses tumor development and metastasis in human breast cancer. Genet Mol Res, 2017. 16(1).

57. Krishnan, P., et al., Next generation sequencing profiling identifies miR-574-3p and miR-660-5p as potential novel prognostic markers for breast cancer. BMC Genomics, 2015. 16: p. 735.

58. Norgen-Biotek-Corp. Total RNA Purification Kit Product Insert Product #17200, 37500, 17250

https://norgenbiotek.com/sites/default/files/resources/Total%20RNA%20Purification%20Kit%20Insert%20PI17200-33.pdf.

59. Busk, P. K., A tool for design of primers for microRNA-specific quantitative RT-qPCR. BMC Bioinformatics, 2014. 15: p. 29.

60. Marabita, F., et al., Normalization of circulating microRNA expression data obtained by quantitative real-time RT-PCR. Brief Bioinform, 2016. 17(2): p. 204-12.

61. Tutz, G. and H. Binder, Generalized additive modeling with implicit variable selection by likelihood-based boosting. Biometrics, 2006. 62(4): p. 961-71.

62. R-Core-Team, R: A language and environment for statistical computing https://www.r-prject.org/.2018.

63. Ghosh, R. K., T. Pandey, and P. Dey, Liquid biopsy: A new avenue in pathology. Cytopathology, 2019. 30(2): p. 138-143.

64. Mader, S. and K. Pantel, Liquid Biopsy: Current Status and Future Perspectives. Oncol Res Treat, 2017. 40(7-8): p. 404-408.

65. Pantel, K. and C. Alix-Panabieres, Liquid biopsy and minimal residual disease—latest advances and implications for cure. Nat Rev Clin Oncol, 2019. 16(7): p. 409-424.

66. Rossi, G. and M. Ignatiadis, Promises and Pitfalls of Using Liquid Biopsy for Precision Medicine. Cancer Res, 2019. 79(11): p. 2798-2804.

67. Alimirzaie, S., M. Bagherzadeh, and M. R. Akbari, Liquid biopsy in breast cancer: A comprehensive review. Clin Genet, 2019. 95(6): p. 643-660.

68. Wang, H. X. and G. Olivier, Tumor-derived extracellular vesicles in breast cancer: from bench to bedside. Cancer Lett, 2019.

69. Zubor, P., et al., Why the Gold Standard Approach by Mammography Demands Extension by Multiomics? Application of Liquid Biopsy miRNA Profiles to Breast Cancer Disease Management. Int J Mol Sci, 2019. 20(12).

70. Beretov, J., et al., Proteomic Analysis of Urine to Identify Breast Cancer Biomarker Candidates Using a Label-Free LC-MS/MS Approach. PLoS One, 2015. 10(11): p. e0141876.

71. Burton, C., et al., Urinary metallomics as a novel biomarker discovery platform: Breast cancer as a case study. Clin Chim Acta, 2016. 452: p. 142-8.

72. Guo, C., et al., Discriminating patients with early-stage breast cancer from benign lesions by detection of oxidative DNA damage biomarker in urine. Oncotarget, 2017. 8(32): p. 53100-53109.

73. Herman-Saffar, O., et al., Early non-invasive detection of breast cancer using exhaled breath and urine analysis. Comput Biol Med, 2018. 96: p. 227-232.

74. Woollam, M., et al., Detection of Volatile Organic Compounds (VOCs) in Urine via Gas Chromatography-Mass Spectrometry QTOF to Differentiate Between Localized and Metastatic Models of Breast Cancer. Sci Rep, 2019. 9(1): p. 2526.

75. Hofbauer, S. L., et al., A urinary microRNA (miR) signature for diagnosis of bladder cancer. Urol Oncol, 2018. 36(12): p. 531 e1-531 e8.

76. Kutwin, P., et al., Urine miRNA as a potential biomarker for bladder cancer detection—a meta-analysis. Cent European J Urol, 2018. 71(2): p. 177-185.

77. Lekchnov, E. A., et al., Searching for the Novel Specific Predictors of Prostate Cancer in Urine: The Analysis of 84 miRNA Expression. Int J Mol Sci, 2018. 19(12).

78. Moldovan, L., et al., Methodological challenges in utilizing miRNAs as circulating biomarkers. J Cell Mol Med, 2014. 18(3): p. 371-90.

79. Liu, J., et al., Tumour-suppressive microRNA-424-5p directly targets CCNE1 as potential prognostic markers in epithelial ovarian cancer. Cell Cycle, 2018. 17(3): p. 309-318.

80. Wei, S., et al., miR-424-5p promotes proliferation of gastric cancer by targeting Smad3 through TGF-beta signaling pathway. Oncotarget, 2016. 7(46): p. 75185-75196.

81. Wu, K., et al., MicroRNA-424-5p suppresses the expression of SOCS6 in pancreatic cancer. Pathol Oncol Res, 2013. 19(4): p. 739-48.

82. Xu, J., et al., Suppressed miR-424 expression via upregulation of target gene Chk1 contributes to the progression of cervical cancer. Oncogene, 2013. 32(8): p. 976-87.

83. Xie, D., et al., MicroRNA424 serves an antioncogenic role by targeting cyclindependent kinase 1 in breast cancer cells. Oncol Rep, 2018. 40(6): p. 3416-3426.

84. Shivapurkar, N., et al., Circulating microRNAs in patients with hormone receptor-positive, metastatic breast cancer treated with dovitinib. Clin Transl Med, 2017. 6(1): p. 37.

85. Fortunato, O., et al., Mir-660 is downregulated in lung cancer patients and its replacement inhibits lung tumorigenesis by targeting MDM2-p53 interaction. Cell Death Dis, 2014. 5: p. e1564.

86. He, T., et al., miR6605p is associated with cell migration, invasion, proliferation and apoptosis in renal cell carcinoma. Mol Med Rep, 2018. 17(1): p. 2051-2060.

87. He, X. and Y. Shu, RNA N6-methyladenosine modification participates in miR-660/E2F3 axis-mediated inhibition of cell proliferation in gastric cancer. Pathol Res Pract, 2019. 215(6): p. 152393.

88. Borzi, C., et al., mir-660-p53-mir-486 Network: A New Key Regulatory Pathway in Lung Tumorigenesis. Int J Mol Sci, 2017. 18(1).

89. Zhang, P., et al., Downregulation of microRNA660 inhibits cell proliferation and invasion in osteosarcoma by directly targeting forkhead box O1. Mol Med Rep, 2018. 18(2): p. 2433-2440.

90. Zhou, C., et al., Combination of serum miRNAs with Cyfra21-1 for the diagnosis of non-small cell lung cancer. Cancer Lett, 2015. 367(2): p. 138-46.

91. Elkhadragy, L., et al., A regulatory BMI1/let-7i/ERK3 pathway controls the motility of head and neck cancer cells. Mol Oncol, 2017. 11(2): p. 194-207.

92. Gadducci, A., et al., Micro-RNAs and ovarian cancer: the state of art and perspectives of clinical research. Gynecol Endocrinol, 2014. 30(4): p. 266-71.

93. Hur, K., et al., Identification of a metastasis-specific MicroRNA signature in human colorectal cancer. J Natl Cancer Inst, 2015. 107(3).

94. Qin, M. M., et al., let-7i inhibits proliferation and migration of bladder cancer cells by targeting HMGA1. BMC Urol, 2019. 19(1): p. 53.

95. Xie, J., et al., miR-7 inhibits the invasion and metastasis of gastric cancer cells by suppressing epidermal growth factor receptor expression. Oncol Rep, 2014. 31(4): p. 1715-22.

96. Yang, W. H., et al., Repression of bone morphogenetic protein 4 by let-7i attenuates mesenchymal migration of head and neck cancer cells. Biochem Biophys Res Commun, 2013. 433(1): p. 24-30.

97. Mahn, R., et al., Circulating microRNAs (miRNA) in serum of patients with prostate cancer. Urology, 2011. 77(5): p. 1265 e9-16.

98. Xiao, D., et al., Melanoma cell-derived exosomes promote epithelial-mesenchymal transition in primary melanocytes through paracrine/autocrine signaling in the tumor microenvironment. Cancer Lett, 2016. 376(2): p. 318-27.

99. Chakraborty, C. and S. Das, Profiling cell-free and circulating miRNA: a clinical diagnostic tool for different cancers. Tumour Biol, 2016. 37(5): p. 5705-14.

100. Zhu, Y., et al., Identification of a serum microRNA expression signature for detection of lung cancer, involving miR-23b, miR-221, miR-148b and miR-423-3p. Lung Cancer, 2017. 114: p. 6-11.

101. Du, W., Z. Feng, and Q. Sun, LncRNA LINC00319 accelerates ovarian cancer progression through miR-423-5p/NACC1 pathway. Biochem Biophys Res Commun, 2018. 507(1-4): p. 198-202.

102. Lin, H., et al., Inhibition of miR-423-5p suppressed prostate cancer through targeting GRIM-19. Gene, 2019. 688: p. 93-97.

103. Yang, H., et al., Exosomal miR-423-5p targets SUFU to promote cancer growth and metastasis and serves as a novel marker for gastric cancer. Mol Carcinog, 2018. 57(9): p. 1223-1236.

104. McDermott, A. M., et al., Identification and validation of oncologic miRNA biomarkers for luminal A-like breast cancer. PLoS One, 2014. 9(1): p. e87032.

105. Ding, H. X., et al., MiRNA Polymorphisms and Cancer Prognosis: A Systematic Review and Meta-Analysis. Front Oncol, 2018. 8: p. 596.

106. Campos-Parra, A. D., et al., Micro-RNAs as Potential Predictors of Response to Breast Cancer Systemic Therapy: Future Clinical Implications. Int J Mol Sci, 2017. 18(6).

107. Liu, B., et al., Serum miR-21 and miR-125b as markers predicting neoadjuvant chemotherapy response and prognosis in stage II/III breast cancer. Hum Pathol, 2017. 64: p. 44-52.

108. Xie, X., et al., The role of miR-125b-mitochondria-caspase-3 pathway in doxorubicin resistance and therapy in human breast cancer. Tumour Biol, 2015. 36(9): p. 7185-94.

109. Hong, L., et al., miR-125b inhibited epithelial-mesenchymal transition of triple-negative breast cancer by targeting MAP2K7. Onco Targets Ther, 2016. 9: p. 2639-48.

110. Matamala, N., et al., Tumor microRNA expression profiling identifies circulating microRNAs for early breast cancer detection. Clin Chem, 2015. 61(8): p. 1098-106.

111. Das, R., et al., MicroRNA-194 Promotes Prostate Cancer Metastasis by Inhibiting SOCS2. Cancer Res, 2017. 77(4): p. 1021-1034.

112. Kong, Q., et al., MicroRNA-194 suppresses prostate cancer migration and invasion by downregulating human nuclear distribution protein. Oncol Rep, 2017. 37(2): p. 803-812.

113. Peng, Y., et al., MiRNA-194 activates the Wnt/beta-catenin signaling pathway in gastric cancer by targeting the negative Wnt regulator, SUFU. Cancer Lett, 2017. 385: p. 117-127.

114. Zhang, X., et al., MicroRNA-194 represses glioma cell epithelialtomesenchymal transition by targeting Bmi1. Oncol Rep, 2017. 37(3): p. 1593-1600.

115. Chen, Y., et al., Promotional effect of microRNA-194 on breast cancer cells via targeting F-box/WD repeat-containing protein 7. Oncol Lett, 2018. 15(4): p. 4439-4444.

116. Gilles, M. E. and F. J. Slack, Let-7 microRNA as a potential therapeutic target with implications for immunotherapy. Expert Opin Ther Targets, 2018. 22(11): p. 929-939.

117. Chernyi, V. S., et al., Search of MicroRNAs Regulating the Receptor Status of Breast Cancer In Silico and Experimental Confirmation of Their Expression in Tumors. Bull Exp Biol Med, 2017. 163(5): p. 655-659.

118. Sueta, A., et al., Differential expression of exosomal miRNAs between breast cancer patients with and without recurrence. Oncotarget, 2017. 8(41): p. 69934-69944.

119. Zavesky, L., et al., Supernatant versus exosomal urinary microRNAs. Two fractions with different outcomes in gynaecological cancers. Neoplasma, 2016. 63(1): p. 121-32.

120. Armand-Labit, V. and A. Pradines, Circulating cell-free microRNAs as clinical cancer biomarkers. Biomol Concepts, 2017. 8(2): p. 61-81.

121. Nedaeinia, R., et al., Circulating exosomes and exosomal microRNAs as biomarkers in gastrointestinal cancer. Cancer Gene Ther, 2017. 24(2): p. 48-56.

122. Pritchard, C. C., et al., Blood cell origin of circulating microRNAs: a cautionary note for cancer biomarker studies. Cancer Prey Res (Phila), 2012. 5(3): p. 492-497.

123. Zhang, J., et al., Exosome and exosomal microRNA: trafficking, sorting, and function. Genomics Proteomics Bioinformatics, 2015. 13(1): p. 17-24.

124. Larssen, P., et al., Tracing Cellular Origin of Human Exosomes Using Multiplex Proximity Extension Assays. Mol Cell Proteomics, 2017. 16(3): p. 502-511.

### Example 2

### Materials and Methods

### Specimen Origin

Human body fluids including serum, urine, ascites, and amniotic liquor served as matrices to test the filter-based microvesicle isolation method. The entirety of human specimen samples used in this study, originate from surplus material of either routine medical interventions or voluntary donators.

### Specimen Pre-Treatment and Storage Conditions

Liquid biopsy specimen were obtained according to the WMA declaration of Helsinki [30]. In general, body fluid specimen were collected and stored in sterile sampling tubes at −20° C. until further processing. Additional pre-treatment steps and detailed information are described for each sample matrix in the following.

Serum: Blood samples were collected by using S-Monovette Serum Gel (#04.1925, Sarstedt AG, Nuembrecht, Germany) and allowed to clot at room temperature for 20 min followed by centrifugation at 2500 g for 10 min at 20° C. to obtain pure serum. Serum samples were transferred to a fresh 15 ml poly propylene (pp) tube (#62.554.502; Sarstedt AG) and stored at −20° C. until further processing.

Urine: Patients provided urine specimen collected in a sterile 70 ml screw-on cap urine sampling tube (#75.9922.745, Sarsted AG). Urine samples were transferred and aliquoted to 10 ml Urine Monovette (#10.252; Sarsted AG) and stored at −20° C. until further processing.

### Microvesicle Isolation

### Pre-Treatment Following Storage

Fresh frozen liquid biopsy samples were thawed at room temperature and centrifuged at 4000 g for 15 min to remove sediment and cellular debris. Following centrifugation, supernatant was transferred in a new 15 ml pp tube (Sarstedt AG) for further processing.

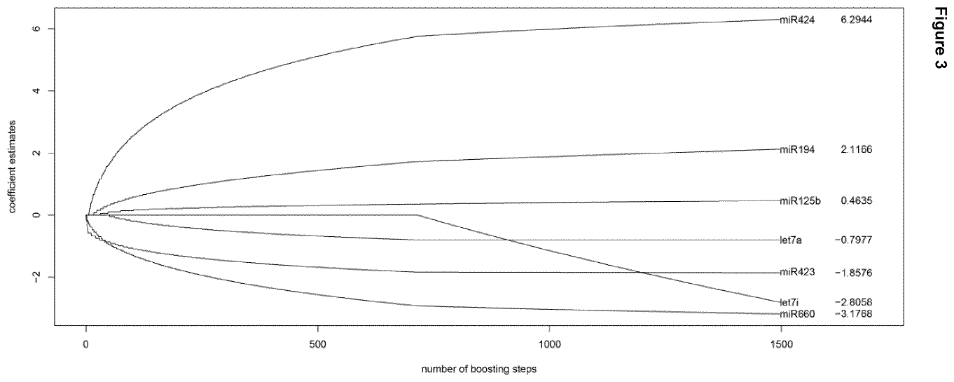

Due to the higher viscosity and protein content, the specimen matrices serum, ascites and amniotic liquor were diluted with sterile 1× Dulbecco's phosphate-buffered saline (DPBS, #14190185; ThermoFisher Scientific, Karlsruhe, Germany) in ratio 2:3 (2 ml sample:3 ml DPBS) to facilitate the filtration process. Sample matrix-dependent methodical variations in workflow are visualized in FIG. 4.

### Arrangement of Equipment and General Processing

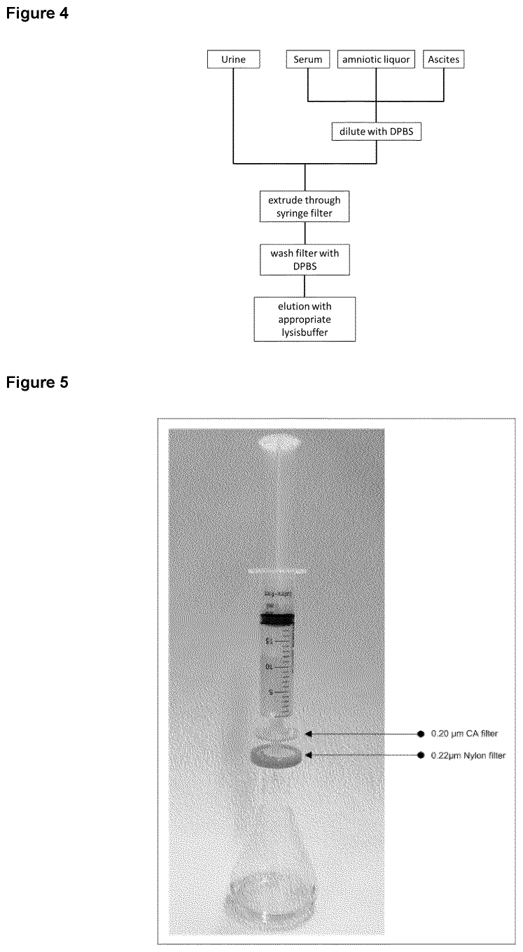

Pre-treated sample liquids were transferred individually to a 20 ml syringe (#4617207V; BRAUN, Melsungen, Germany) with a screwed on 0.2 μm cellulose acetate (CA) syringe filter (#514-0061; VWR International GmbH, Darmstadt, Germany) followed by a second screwed on 0.22 μm nylon syringe filter (#02542904; Perkin Elmer, Waltham, USA). Upon insertion of the piston, the individual samples were filtered dropwise through the screwed on filters (FIG. 5). Following filtration of the complete sample volume, the cascaded filters were washed with 5 ml 1× DPBS (Thermo) using a fresh 5 ml syringe (#4617053V; BRAUN, Melsungen, Germany). Washing guarantees the removal of sample residues falling below preset filter limitation that possibly interfere with downstream applications. Rinsed filters were then separated.

The CA filter retained all particles >200 nm, including microvesicles within the respective molecular size limit. Due to the non-protein binding characteristics of the upstream CA filter, all microvesicles <200 nm flow through and subsequently bound on the protein binding nylon membrane of the second filter.

Elution of the two different filter-bound microvesicle fractions was carried out by extruding 500 μl protein lysis buffer (100mM HEPES pH 7.2, 0.1% SDS, 0.1% TRITON X100) through each of the used separate filters with a new 2 ml syringe.

### Sample Matrix-Specific Processing

For isolation of urinary microvesicles 20 ml centrifuged urine/sample were utilized in the filtration step with satisfying yield independent from inter-sample concentration differences.

Serum, ascites and amniotic liquor specimen were diluted with DPBS as fore cited and a final volume of 5 ml was filtrated to harvest the two different size fractions of microvesicles.

### Western Blot

Protein concentrations of specimen isolates were quantified utilizing BCA method (#23227; ThermoFisher Scientific, Karlsruhe, Germany). Protein extracts obtained via the novel isolation method were analyzed by Western blot technique. 10 μg of total protein were separated by SDS-PAGE and transferred to an IMMOBILON-P membrane (#IPVH00010; Merck, Darmstadt, Germany) by using a Mini PROTEAN tetra cell (BioRad, München, Germany). Following transfer, membranes were blocked with 3% skim milk in PBS-T and subsequently incubated with the primary antibody (AnnexinV #8555, Flotillin1 #18634S, HSP70 #46477S, CD9 #13174S CellSignaling Technology, β-Actin #SC-69879 Santa Cruz Biotechnology) over night at 4° C.

Primary antibody was rinsed by two washing steps with PBS-T, before membranes were incubated with the secondary antibody (anti-rabbit HRP #7074S, CellSignaling Technology, anti-mouse HRP #115-036-003, Jackson ImmunoResearch Europe) for 1 hour at room temperature. Incubation with secondary antibody was stopped by triplicate membrane washing with PBS-T.

ECL reaction was carried out according to Haan & Behrmann [31] . X-Ray films (Super RX-N, Fujifilm Europe, Dusseldorf, Germany) were processed utilizing the AGFA CP1000 X-Ray film processor.

### Results

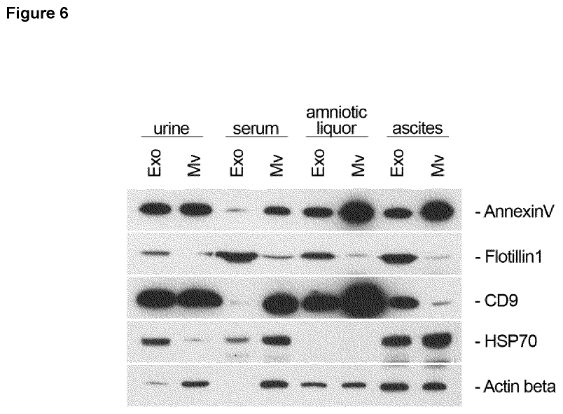

The results are depicted in the Western Blot shown in FIG. 6.

### REFERENCES ONLY FOR EXAMPLE 2

1. Kreger, B. T., et al., Microvesicle Cargo and Function Changes upon Induction of Cellular Transformation. J Biol Chem, 2016. 291(38): p. 19774-85.

2. Tricarico, C., J. Clancy, and C. D'Souza-Schorey, Biology and biogenesis of shed microvesicles. Small GTPases, 2017. 8(4): p. 220-232.

3. Colombo, M., G. Raposo, and C. Thery, Biogenesis, secretion, and intercellular interactions of exosomes and other extracellular vesicles. Annu Rev Cell Dev Biol, 2014. 30: p. 255-89.

4. Bakhshandeh, B., M. A. Kamaleddin, and K. Aalishah, A Comprehensive Review on Exosomes and Microvesicles as Epigenetic Factors. Curr Stem Cell Res Ther, 2017. 12(1): p. 31-36.

5. Vitkova, V., J. Zivny, and J. Janota, Endothelial cell-derived microvesicles: potential mediators and biomarkers of pathologic processes. Biomark Med, 2018. 12(2): p. 161-175.

6. van der Pol, E., et al., Recent developments in the nomenclature, presence, isolation, detection and clinical impact of extracellular vesicles. J Thromb Haemost, 2016. 14(1): p. 48-56.

7. Kowal, J., et al., Proteomic comparison defines novel markers to characterize heterogeneous populations of extracellular vesicle subtypes. Proc Natl Acad Sci U S A, 2016. 113(8): p. E968-77.

8. Cocucci, E. and J. Meldolesi, Ectosomes and exosomes: shedding the confusion between extracellular vesicles. Trends Cell Biol, 2015. 25(6): p. 364-72.

9. Minciacchi, V. R., M. R. Freeman, and D. Di Vizio, Extracellular vesicles in cancer: exosomes, microvesicles and the emerging role of large oncosomes. Semin Cell Dev Biol, 2015. 40: p. 41-51.

10. Gopal, S. K., et al., Extracellular vesicles: their role in cancer biology and epithelial-mesenchymal transition. Biochem J, 2017. 474(1): p. 21-45.

11. Xu, R., et al., Extracellular vesicle isolation and characterization: toward clinical application. J Clin Invest, 2016. 126(4): p. 1152-62.

12. Chen, Y., et al., Extracellular vesicles as novel biomarkers and pharmaceutic targets of diseases. Acta Pharmacol Sin, 2018. 39(4): p. 499-500.

13. Valencia, K. and F. Lecanda, Microvesicles: Isolation, Characterization for In Vitro and In Vivo Procedures. Methods Mol Biol, 2016. 1372: p. 181-92.

14. Erdbrugger, U. and J. Lannigan, Analytical challenges of extracellular vesicle detection: A comparison of different techniques. Cytometry A, 2016. 89(2): p. 123-34.

15. Choi, D. S., et al., Proteomics of extracellular vesicles: Exosomes and ectosomes. Mass Spectrom Rev, 2015. 34(4): p. 474-90.

16. Choi, D. S., et al., Proteomics, transcriptomics and lipidomics of exosomes and ectosomes. Proteomics, 2013. 13(10-11): p. 1554-71.

17. Enderle, D., et al., Characterization of RNA from Exosomes and Other Extracellular Vesicles Isolated by a Novel Spin Column-Based Method. PLoS One, 2015. 10(8): p. e0136133.

18. Chiriaco, M. S., et al., Lab-on-Chip for Exosomes and Microvesicles Detection and Characterization. Sensors (Basel), 2018. 18(10).

19. Szatanek, R., et al., Isolation of extracellular vesicles: Determining the correct approach (Review). Int J Mol Med, 2015. 36(1): p. 11-7.

20. Yuana, Y., et al., Handling and storage of human body fluids for analysis of extracellular vesicles. J Extracell Vesicles, 2015. 4: p. 29260.

21. Giebel, B. and C. Helmbrecht, Methods to Analyze EVs. Methods Mol Biol, 2017. 1545: p. 1-20.

22. Keerthikumar, S., et al., Bioinformatics Tools for Extracellular Vesicles Research. Methods Mol Biol, 2017. 1545: p. 189-196.

23. Yuana, Y., A. Sturk, and R. Nieuwland, Extracellular vesicles in physiological and pathological and pathological conditions. Blood Rev, 2013. 27(1): p. 31-9.

24. Boing, A. N., et al., Single-step isolation of extracellular vesicles by size-exclusion chromatography. J Extracell Vesicles, 2014. 3.

25. Coumans, F. A., et al., Reproducible extracellular vesicle size and concentration determination with tunable resistive pulse sensing. J Extracell Vesicles, 2014. 3: p. 25922.

26. Gray, W. D., A. J. Mitchell, and C. D. Searles, An accurate, precise method for general labeling of extracellular vesicles. MethodsX, 2015. 2: p. 360-7.

27. Mrvar-Brecko, A., et al., Isolated microvesicles from peripheral blood and body fluids as observed by scanning electron microscope. Blood Cells Mol Dis, 2010. 44(4): p. 307-12.

28. Sun, Y., et al., Facile preparation of salivary extracellular vesicles for cancer proteomics. Sci Rep, 2016. 6: p. 24669.

29. Yuana, Y., et al., Co-isolation of extracellular vesicles and high-density lipoproteins using density gradient ultracentrifugation. J Extracell Vesicles, 2014. 3.

30. The World Medical Association, I. WMA Declaration of Helsinki—Ethical Principles for Medical Research Involving Human Subjects 2018 [cited 2018; Available from:

https:www.wma.net/policies-post/wma-declaration-of-helsinki-ethical-principles-for-medical-research-involving-human-subjects/.

31. Haan, C. and I. Behrmann, A cost effective non-commercial ECL-solution for Western blot detections yielding strong signals and low background. J Immunol Methods, 2007. 318(1-2): p. 11-9.

1. A method for enriching or isolating extracellular vesicles from a body fluid sample, said method comprising the steps of: (i) optionally filtrating the body fluid sample through a first membrane, wherein said first membrane has a pore size ranging from 180 nm to 250 nm and docs not bind proteins, (ii) filtrating the body fluid sample or, if step (i) is carried out, the filtrate obtained from step (i) through a second membrane, wherein said second membrane has a pore size ranging from 180 nm to 250 nm and is a protein-binding membrane; and (iii) recovering the extracellular vesicles from the second membrane.

2. The method of claim 1, wherein the vesicles are selected from the group consisting of microvcsiclcs and exosomes.

3. The method of claim 1, wherein the body fluid is blood, plasma, urine, ascites, saliva, or amniotic liquor.

4. The method of claim 1, wherein the body fluid is urine.

5. The method of claim 1, wherein the first membrane comprises cellulose acetate.

6. The method of claim 1, wherein the second membrane comprises a polyamide.

7. The method of claim 1, wherein the pore size of the first membrane is about 0.20 μm.

8. The method of claim 1, wherein the pore size of the second membrane is about 0.22 μm.

9. The method of claim 1, wherein the body fluid sample is subjected to a centrifugation step prior to step (ii), or, if step (i) is carried out, prior to step (i).

10. The method of claim 9, wherein said centrifugation step comprises centrifuging the body fluid sample at 3,000 g to 5,000 g for 5 to 30 minutes.

11. The method of claim 1, wherein said recovering of step (iii) is effected by contacting the second membrane with a lysis buffer.

12. The method of claim 11, wherein said lysis buffer comprises at least one surfactant.

13. The method of claim 12, wherein said at least one surfactant is selected from the group consisting of sodium lauryl sulfate (SDS), polyethylene glycol p-(1,1,3,3-tetramethylbutyl)-phenyl ether (Triton X100) and combinations thereof.

14. The method of claim 1, wherein said method further comprises the step of separating microvesicles from exosomes.

15. A method of diagnosing cancer in a subject said method comprising the steps of: (i) determining the level of miR-424, miR-660 and let7-i in a urine sample obtained from said subject, wherein said urine sample is first enriched in extracellular vesicles by the method of claim 1, (ii) comparing the level determined in step (i) to the level of the respective miR gene products in a control sample, and (iii) diagnosing said subject with cancer when the level of miR-424, miR-660 and let7-i determined in step (i) differs front the associated control level.

16. The method of claim 15, wherein said method further comprises the steps of: (i) determining the level of miR-423, miR-125b or let7-d in the enriched urine sample obtained from said subject, (ii) comparing the level determined in step (i) to the level of the respective miR gene products in the control sample, and (iii) diagnosing said subject with cancer when the level of miR-423, miR-125b or let7-d determined in step (i) differs from the associated control level.

17. The method of claim 15, wherein said method further comprises the step of: (i) determining the level of let7-f, miR-222, miR-194 and/or miR-17 in the enriched urine sample obtained from the subject, (ii) comparing the level determined in step (i) to the level of the respective miR gene products in the control sample, and (iii) diagnosing said subject with cancer when the level of let7-f, miR-222, miR-194 and/or miR-17 determined in step (i) differs from the associated control level.

18. A method of diagnosing cancer in a subject, said method comprising the steps of:
a) determining the level of miR-424, miR-660 and let7-i in a urine sample obtained from the subject, wherein said urine sample is first enriched in extracellular vesicles by the method of claim 1.
b) comparing the level of miR-424 in the urine sample to a control level of miR-424, comparing the level of miR-660 in the urine sample to a control level of miR-660, and comparing the level of let7-i in the urine sample to a control level of let7-i; and
c) diagnosing the subject with cancer when (i) a decrease in the level of miR-660 in the urine sample, relative to the control level of miR-660, (ii) a decrease in the level of let7-i in the urine sample, relative to the control level of let7-i, and (iii) an increase in the level of miR-424 in the urine sample, relative to the control level of miR-424, is detected.

19. The method of claim 18, said method further comprising the steps of:
a) determining the level of miR-423, miR-125b or let7-d in the enriched urine sample front the subject,
b) comparing the level of miR-423, miR-125b or let7-d in the urine sample to the control level of the respective miR gene product, and
c) diagnosing the subject with cancer when an increase in the level of miR-125b or let7-d in the urine sample, relative to the respective control level of miR-125b or let7-d, or a decrease in the level of miR-423 in the urine sample, relative to the control level of miR-423, is detected.

20. The method of claim 18, said method further comprising:
a) determining the level of miR-17, let7-f, miR-222 and/or miR-194 in the enriched urine sample from the subject,
b) comparing the level of miR-17, let7-f, miR-222 and/or miR-194 in the urine sample to a control level of the respective miR gene product, and
c) diagnosing the subject with cancer when an increase in the level of miR-17, miR-222 and/or miR-194 in the urine sample, relative to the respective control level of miR-17, miR-222 or miR-194, and/or a decrease in the level of let7-f, relative to the control level of let7-f, is detected.

21. The method of claim 15, wherein the control sample is a urine sample from one or more healthy individuals.

22. The method of claim 15, wherein the subject is a female human.

23. The method of claim 15, wherein the level of miR gene product(s) is measured by quantitative RT-PCR, digital PCR, next generation sequencing, or by hybridisation using an oligonucleotide microarray.

24. The method of claim 15, wherein a measured decrease of at least 25% in the levels of miR-660 and let7-i in the urine sample from the subject, relative to the respective control levels of the respective gene products, and/or a measured increase of at least 25% in the level of miR-424, in the urine sample from the subject, relative to the control level of miR-424 gene product, indicates the presence of breast cancer.

25. A kit for the detection or diagnosis of breast cancer in a subject, said kit comprising: (i) an oligonucleotide capable of hybridizing to a nucleic acid consisting of SEQ ID NO:11, (ii) an oligonucleotide capable of hybridizing to a nucleic acid consisting of SEQ ID NO:12, and (iii) an oligonucleotide capable of hybridizing to a nucleic acid consisting of SEQ ID NO:17; wherein said kit may optionally further comprise (iv) an oligonucleotide capable of hybridizing to a nucleic acid consisting of SEQ ID NO:7, (v) an oligonucleotide capable of hybridizing to a nucleic acid consisting of SEQ ID NO:10 and/or (vi) an oligonucleotide capable of hybridizing to a nucleic acid consisting of SEQ ID NO:14.

26. (canceled)

27. (canceled)
